(19) United States
(12) Patent Application Publication
(10) Pub. No.: US 2017/0226561 A1
(43) Pub. Date: Aug. 10, 2017

(54) MAPPING THE SPATIAL LOCALIZATION OF CELLULAR NUCLEIC ACIDS BY PROXIMITY-DEPENDENT ENZYMATIC TAGGING

(71) Applicants: The Board of Trustees of the Leland Stanford Junior University, Stanford, CA (US); Massachusetts Institute of Technology, Cambridge, MA (US)

(72) Inventors: Alice Ting, Stanford, CA (US); Pornchai Kaewsapsak, Stanford, CA (US)

(21) Appl. No.: 15/424,830

(22) Filed: Feb. 4, 2017

Related U.S. Application Data
(60) Provisional application No. 62/291,189, filed on Feb. 4, 2016.

(51) Int. Cl. C12Q 1/68

(52) U.S. Cl. CPC C12Q 1/686; C12Q 1/6804

(57) ABSTRACT

Compositions and methods for mapping the spatial localization of cellular nucleic acids by proximity-dependent enzymatic tagging are described. In particular, proximity-specific biotinylation of nucleic acids is combined with sequencing to identify nucleic acids, including DNA or RNA molecules in proximity to a protein of interest or within or near a particular subcellular compartment in vivo.

### CROSS-REFERENCE TO RELATED APPLICATION

This application claims benefit under 35 U.S.C. §119(e) of provisional application 62/291,189, filed Feb. 4, 2016, which application is hereby incorporated by reference in its entirety.

### TECHNICAL FIELD

The present invention pertains generally to methods of mapping subcellular localization of nucleic acids. In particular, the invention relates to compositions and methods for proximity-specific tagging of nucleic acids to identify nucleic acids, including DNA or RNA molecules in proximity to a protein of interest or within or near a particular subcellular compartment in vivo.

### BACKGROUND

Ribonucleic acids (RNAs) comprise a diverse class of biomolecules that participate in a staggering breadth of fundamental processes in all living cells. Although, based on a small handful of examples, it has been speculated that subcellular localization may generally be a critical determinant of RNA function, current methods that identify the location of RNAs en masse have proven cumbersome, low-throughput, difficult and noisy. Most existing technologies for studying RNA localization are either based on microscopic fluorescence imaging, or require native purification of the target subcellular compartment in vitro. Methods in the former category are often extremely low-throughput (i.e. allowing only a handful of RNAs to be analyzed at a time), or alternatively require highly specialized next-generation microscopic equipment and/or a large array of custom biochemical reagents. Methods in the latter category require the development of a robust purification scheme for the target compartment, which may entail substantial loss of loosely affiliated RNAs, or may generally be impossible. In both cases, separating the biological signal from experimental noise can be extremely challenging.

Thus, there remains a need for a better, efficient, high-throughput methods of determining RNA localization.

### SUMMARY

The present invention is based, in part, on the discovery of a new method for determining subcellular localization of nucleic acids, including RNA and DNA. In particular, the invention relates to a method combining proximity-specific labeling of nucleic acids with sequencing to identify nucleic acids within or near a particular subcellular compartment in vivo.

In one aspect, the invention includes method of mapping subcellular localization of nucleic acids in a cell, the method comprising: a) introducing a tagging enzyme into the cell, wherein the tagging enzyme is targeted to a subcellular region of interest; b) contacting the cell with a tagging substrate for the tagging enzyme, wherein the tagging enzyme catalyzes a reaction with the tagging substrate resulting in covalent attachment of a tag to nucleic acids within an intracellular spatial location around the tagging enzyme; and c) isolating the tagged nucleic acids using an agent that selectively binds to the tag; and c) analyzing the tagged nucleic acids to produce a map of the subcellular localization of the nucleic acids.

In certain embodiments, the tagged nucleic acids are isolated using an agent, such as an antibody, a probe, a ligand, or an aptamer that selectively binds to the tag. The agent may be immobilized on a solid support, such as, but not limited to, a magnetic bead, latex bead, microtiter plate well, glass plate, nylon, agarose, or acrylamide. In another embodiment, the method further comprises lysing the cell.

In certain embodiments, the tagging enzyme is a peroxidase. Exemplary peroxidases include horseradish peroxidase and ascorbate peroxidase. In one embodiment, the tagging enzyme is an engineered ascorbate peroxidase (e.g., APEX or APEX2). Phenol and phenolic compounds such as tyramine or phenolic aryl azide derivatives react with hydrogen peroxide to generate short lived, reactive free radicals. For example, proximity labeling can be performed in the presence of hydrogen peroxide and biotin-phenol (BP), wherein the peroxidase catalyzes the reaction of the biotin-phenol with the hydrogen peroxide to produce a biotin-phenoxyl radical that reacts with nearby nucleic acids resulting in biotinylation (i.e., tagging) of the nucleic acids.

Biotinylated nucleic acids, produced with a peroxidase, as described herein, can be isolated with a biotin-binding protein, such as streptavidin or avidin.

In another embodiment, the method further comprises treating the cell with a radical quencher (e.g., ascorbate, 6-hydroxy-2,5,7,8-tetramethylchroman-2-carboxylic acid (TROLOX), or sodium azide) after tagging of the nucleic acids.

In certain embodiments, the tagging enzyme comprises a targeting sequence that directs the tagging enzyme to the subcellular region of interest. Exemplary targeting sequences include a secretory protein signal sequence, a membrane protein signal sequence, a nuclear localization sequence, a mitochondrial localization sequence, an outer mitochondrial membrane sequence, an endoplasmic reticulum localization sequence, an endoplasmic reticulum membrane targeting sequence, a nucleolar localization signal sequence, a nuclear export signal sequence, a peroxisome localization sequence, and a protein binding motif sequence. In another embodiment, the targeting sequence comprises a sequence selected from the group consisting of SEQ ID NOS:1-5.

In other embodiments, the tagging enzyme is covalently linked to a peptide or protein that directs the tagging enzyme to the subcellular region of interest, such as a cytosolic protein, a nuclear protein, a membrane protein, a mitochondrial protein, a P-body protein, or a secretory pathway protein.

In another embodiment, introducing the tagging enzyme into the cell comprises transfecting the cell with a recombinant polynucleotide comprising a promoter operably linked to a polynucleotide encoding the tagging enzyme. The recombinant polynucleotide may comprise an expression vector, for example, a bacterial plasmid vector or a viral expression vector, such as, but not limited to, an adenovirus, retrovirus (e.g., γ-retrovirus and lentivirus), poxvirus, adeno-associated virus, baculovirus, or herpes simplex virus vector.

The cell can be any type of cell, including any eukaryotic cell, prokaryotic cell, or archaeon cell. For example, the cell may be an animal cell, plant cell, fungal cell, or protist cell. Alternatively, the cell can be an artificial cell, such as a nanoparticle, liposome, polymersome, or microcapsule encapsulating the nucleic acids.

RNA isolated and mapped by the methods described herein can be animal RNA, bacterial RNA, fungal RNA, protist RNA, or plant RNA. In one embodiment, the RNA is human RNA.

In another embodiment, the method further comprises amplifying at least one RNA or DNA molecule. RNA molecules may be amplified, for example, by performing reverse transcription polymerase chain reaction (RT-PCR).

In another embodiment, the method further comprises sequencing at least one RNA or DNA of the isolated tagged nucleic acids.

In another embodiment, the method further comprises multiplex sequencing of the tagged nucleic acids. For example, sequencing may comprise performing deep sequencing or next-generation sequencing.

In another embodiment, the method further comprises identifying at least one RNA or DNA molecule of the tagged nucleic acids (e.g., of a messenger RNA, a ribosomal RNA, a transfer RNA, a non-coding RNA, or a regulatory RNA).

In another embodiment, the method further comprises identifying at least one ribonucleoprotein (RNP) interaction.

In another embodiment, the method further comprises calculating the frequencies of one or more RNA molecules that are present within the intracellular spatial location.

In another embodiment, the method further comprises quantitating one or more RNA molecules that are present within the intracellular spatial location.

In certain embodiments, the cell is exposed to a test condition prior to said contacting the cell with the tagging substrate. For example, a test condition may comprise exposing the cell to a drug, a ligand for a receptor, a hormone, a second messenger, a pathogen, or a genetic modification. For example, the cell can be genetically modified by introducing a vector, short hairpin RNA (shRNA), small interfering RNA (siRNA), microRNA (miRNA), or CRISPR-associated system into the cell. Alternatively, a test condition may comprise exposing the cell to a change in temperature, growth media, membrane potential, or osmotic pressure.

In certain embodiments, a map of the subcellular localization of the nucleic acids molecules, produced by the methods described herein, is compared to a reference map. For example, a map of the subcellular localization of the RNA molecules from a cell that is exposed to the test condition can be compared to a reference map of a cell that is not exposed to the test condition. In another embodiment, the method further comprises comparing a map of the subcellular localization of the nucleic acid molecules within the intracellular spatial location to a reference map for a cell at the same or a different developmental stage.

These and other embodiments of the subject invention will readily occur to those of skill in the art in view of the disclosure herein.

### BRIEF DESCRIPTION OF THE FIGURES

FIG. 1 shows a scheme of the optimized labeling protocol. Live HEK cells expressing APEX2 in the compartment of interest (in this example, ER membrane facing cytosol) are treated with H₂O₂ in the presence of biotin-phenol for 1 minute (red B=biotin), Then RNAs were extracted and enriched with streptavidin beads. The biotinylated RNAs are eluted and analyzed by qPCR or RNA Seq. The zoom-in panel displays a purposed reaction where the biotin-phenoxyl radical reacts with RNAs through guanine base.

FIG. 2 shows a dot blot analysis of in-vitro biotinylation of tRNA by HRP. tRNA were reacted with BP, H₂O₂ and HRP for 1 minute at room temperature. Omission of each reagent was performed as negative controls. The labeled RNAs were either spotted on the nitrocellulose membrane directly (row A) or treated with none (row B), protease K (row C), or RNase A (row D) prior spotting. The blot was probed with streptavidin-HRP to visualize the presence of biotin moiety.

FIG. 3 shows a dot blot analysis of in-vitro biotinylation of purified cellular RNAs by HRP. Purified cellular RNAs from HEK293T were reacted with BP, H₂O₂ and HRP for 30 minutes at 37° C. Omission of each reagent was performed as negative controls. After treatment with RNase A (bottom row) or mock treatment (top row), the RNAs were spotted on the nitrocellulose membrane. The blot was probed with streptavidin-HRP to visualize the presence of biotin moiety. The in-vitro BP-labeled tRNA and biotin-labeled DNA oligonucleotide were used as positive controls. Bright field was shown to indicate the location of each spot.

FIGS. 4A-4C show a Southern blot analysis of in-vitro biotinylation of purified double-stranded DNA (dsDNA). The dsDNA was reacted with BP, H₂O₂ and HRP for 60 minutes at 37° C. Omission of each reagent was performed as negative controls. The in-vitro BP-labeled tRNA was used as a positive control. The RNAs were resolved in gel and transferred to positively-charged nylon blot. The blot was probed with streptavidin-HRP to visualize the presence of biotin moiety. FIG. 4A shows ethidium bromide (EtBr)-stained gel before transfer to blot. FIG. 4B shows EtBr-stained gel after transfer to the blot. FIG. 4C displays the chemoluminescent signal from streptavidin. The white arrow indicates the biotinylated DNAs in lane 1.

FIG. 5 shows a dot blot analysis of in-vitro biotinylation of purified cellular RNAs by HRP. Purified genomic DNAs from HEK293T were reacted with BP, H₂O₂ and HRP for 30 minutes at 37° C. Omission of each reagent was performed as negative controls. After clean up, the DNAs were spotted on the nitrocellulose membrane. The blot was probed with streptavidin-HRP to visualize the presence of biotin moiety. The biotin-labeled DNA oligonucleotide was used as a positive control. Bright field was shown to indicate the location of each spot.

FIGS. 6A-6D show a Southern blot analysis of in-vitro biotinylation of purified genomic DNA. Genomic DNA purified from HEK293T was reacted with BP, H₂O₂ and HRP for 60 minutes at 37° C. Omission of each reagent was performed as negative controls. The biotin-labeled DNA oligonucleotide was used as a positive control. The RNAs were resolved in gel and transferred to positively-charged nylon blot. The blot was probed with streptavidin-HRP to visualize the presence of biotin modification. FIG. 6A panel shows EtBr-stained gel before transfer to blot. FIG. 6B shows EtBr-stained gel after transfer to the blot. FIG. 6C shows ethidium bromide (EtBr)-stained blot after transfer. FIG. 6D displays the chemoluminescent signal from streptavidin.

FIG. 7 shows a denaturing gel electrophoretic analysis of biotin-phenol labeled 5S rRNA. 5s RNA was reacted with BP, H₂O₂ and HRP for 1 min. The labeled RNAs were enriched with streptavidin magnetic beads. The enriched RNAs were then in-vitro transcribed with P³² primer. The cDNAs were separated in denaturing gel and visualized by phophorimaging. Left: structure of 5s RNA. Blue circles indicate the site of modification. The gray circle denotes the position of RNAs with no data from the gel.

FIG. 8 shows RT-qPCR analysis of mitochondrial RNA enrichment by mito-APEX2. HEK293T expressing mitochondrial matrix-localized APEX (mito-APEX2) was labeled with BP and H₂O₂. The enriched RNAs were quantified by RT-qPCR. SSR2, TMX1, and SFT2P2 represent ER-associated RNAs. FAU, SUB1, and GAPDH represent cytosolic RNAs. MTND1 and MTCO2 represent mitochondrial RNAs. XIST represents nuclear RNA.

FIG. 9 shows RT-qPCR analysis of nuclear RNA enrichment by APEX2-NLS. HEK293T expressing nuclear localized APEX (APEX2-NLS) was labeled with BP and H₂O₂. The enriched RNAs were quantified by RT-qPCR. MTND1 and MTCO2 represent mitochondrial RNAs. NEAT1, MALAT1, and XIST represent nuclear RNA. GAPDH represents cytosolic RNAs.

FIG. 10 shows RT-qPCR analysis of ER-associated RNA enrichment by ERM-APEX2. HEK293T expressing APEX on ER membrane facing cytosol (ERM-APEX2) was labeled with BP and H₂O₂. The enriched RNAs were quantified by RT-qPCR. SSR2, TMX1, and SFT2P2 represent ER-associated RNAs. FAU, SUB 1, and GAPDH represent cytosolic RNAs. MTND1 and MTCO2 represent mitochondrial RNAs. XIST represents nuclear RNA.

FIGS. 11A-11D show RNA-seq analysis of mitochondrial RNA enrichment by mito-APEX2. The labeled RNAs by mito-APEX2 were subjected to RNA-seq analysis. FIG. 11A shows a scatter plot of the RNA-seq. Each detected gene is plotted according to its RNA abundance after streptavidin enrichment in experiment (x axis) and control (omit H₂O₂, y axis). 15 known mitochondrially-encoded RNAs are colored in green. Long non-coding RNAs are colored in red. Other RNAs are colored in black. FIG. 11B shows the distribution of RNAs from RNA-seq according to log 2 FPKM ratio post streptavidin enrichment between experiment and control. The green bars are the known mitochondrial RNAs and red bars are long non-coding RNAs. FIG. 11C shows ROC analysis of mitochondrial RNA dataset. For each FPKM ratio cutoff, the True Positive Rate (TPR) was plotted against the False Positive Rate (FPR). TPR is defined as the fraction of mitochondrial RNAs above the cutoff. FPR is defined as the fraction of long non-coding RNAs above the cutoff. FIG. 11D shows TPR-FPR values are plotted for each FKPM ratio. The log₂ FPKM ratio corresponds to the maximum was as the cutoff.

FIGS. 12A-12C show RNA-seq analysis of ER-associated RNA enrichment by ERM-APEX2. The labeled RNAs by ERM-APEX2 were subjected to RNA-seq analysis. In this figure, only RNAs that have count greater than or equals to 500 are considered. FIG. 12A shows scatter plot of the RNA-seq. Each detected gene is plotted according to its RNA abundance after streptavidin enrichment in experiment (x axis) and control (omit H₂O₂, y axis). ER-associated RNAs by ER proximal ribosome profiling are colored green. Predicted non-secretory RNAs are colored in red. Other RNAs are in black. FIG. 12B shows an alternative view of (FIG. 12A) but other RNAs (black) are plotted on top. FIG. 12C shows the distribution of RNAs from RNA-seq according to log 2 FPKM ratio post streptavidin enrichment between experiment and control. Green bars are ER-associated RNAs by ER proximal ribosome profiling and red bars are Predicted non-secretory RNAs.

FIG. 13 shows an RT-qPCR analysis of mitochondrial-associated RNA enrichment by OMM-APEX2. HEK293T expressing APEX on outer mitochondrial membrane facing cytosol (OMM-APEX2) was labeled with BP and H₂O₂. The enriched RNAs were quantified by RT-qPCR. IARS2, POLG, LARS, LASR2, MRPL27, and TIMM44 represent potential mitochondrial-associated RNAs. SSR2, GAPDH, and MTCO2 represent other RNAs.

FIGS. 14A-14C show optimization of enrichment protocol. The labeled RNAs from mito-APEX2 expressing cells were enriched according to the protocol in the table shown in FIG. 14A. After enrichment, the RNAs were quantified using RT-qPCR for mitochondrial RNAs (MTND1 and MTCO2) and non-mitochondrial RNAs (GAPDH, XIST, FAU and SSR2). FIG. 14B displays the enrichment ratio (average percent recovery of mitochondrial versus non-mitochondrial RNAs) of different methods. FIG. 14C shows the yield of enriched RNAs.

FIGS. 15A and 15B show further optimization of enrichment protocol. The labeled RNAs were enriched with the conditions in the table shown in FIG. 15A. FIG. 15B displays the enrichment ratio (top row) and the yield (bottom row) of enriched RNAs for each protocol. The box indicates the optimal conditions that provide a good enrichment ratio and high yield after enrichment.

FIGS. 16A and 16B show RNA-seq analysis of mitochondrial RNA enrichment by mito-APEX2 using optimized enrichment protocol. The labeled RNAs by mito-APEX2 were enriched using optimized protocol. The enriched RNA was subjected to RNA-seq analysis. FIG. 16A shows RNA-seq results from the sequencing library without any rRNA depletion. Each detected gene is plotted according to its RNA abundance after streptavidin enrichment in control (omit H₂O₂, x axis) and experiment (y axis). 15 known mitochondrially-encoded RNAs are colored in light gray. Other RNAs are colored in black. FIG. 16B shows RNA-seq results from the sequencing library with polyA selection.

### DETAILED DESCRIPTION

The practice of the present invention will employ, unless otherwise indicated, conventional methods of pharmacology, chemistry, biochemistry, recombinant DNA techniques and immunology, within the skill of the art. Such techniques are explained fully in the literature. See, e.g., A. L. Lehninger, Biochemistry (Worth Publishers, Inc., current addition); Sambrook, et al., Molecular Cloning: A Laboratory Manual (3^{rd} Edition, 2001); RNA: Methods and Protocols (Methods in Molecular Biology, edited by H. Nielsen, Humana Press, 1st edition, 2010); Rio et al. RNA: A Laboratory Manual (Cold Spring Harbor Laboratory Press; 1st edition, 2010); Farrell RNA Methodologies: Laboratory Guide for Isolation and Characterization (Academic Press; 4^{th} edition, 2009); Methods In Enzymology (S. Colowick and N. Kaplan eds., Academic Press, Inc.).

All publications, patents and patent applications cited herein, whether supra or infra, are hereby incorporated by reference in their entireties.

### I. Definitions

In describing the present invention, the following terms will be employed, and are intended to be defined as indicated below.

It must be noted that, as used in this specification and the appended claims, the singular forms “a,” “an” and “the” include plural referents unless the content clearly dictates otherwise. Thus, for example, reference to “an RNA” includes a mixture of two or more RNA, and the like.

The term “about,” particularly in reference to a given quantity, is meant to encompass deviations of plus or minus five percent.

As used herein, a “cell” refers to any type of cell isolated from a prokaryotic, eukaryotic, or archaeon organism, including bacteria, archaea, fungi, protists, plants, and animals, including cells from tissues, organs, and biopsies, as well as recombinant cells, cells from cell lines cultured in vitro, and cellular fragments, cell components, or organelles comprising nucleic acids. The term also encompasses artificial cells, such as nanoparticles, liposomes, polymersomes, or microcapsules encapsulating nucleic acids. A cell may include a fixed cell or a live cell. The methods described herein can be performed, for example, on a sample comprising a single cell or a population of cells.

A “live cell,” as used herein, refers to an intact cell, naturally occurring or modified. The live cell may be isolated from other cells, mixed with other cells in a culture, or within a tissue (partial or intact) or an organism. In some embodiments, the live cell is a cell engineered to express a tagging enzyme, for example, a peroxidase. In some embodiments, the live cell expresses a tagging enzyme that is targeted to a subcellular compartment or structure, for example, via a localization signal within or fused to the tagging enzyme.

The terms “nucleic acid,” “nucleic acid molecule,” “polynucleotide,” and “oligonucleotide” are used herein to include a polymeric form of nucleotides of any length, either ribonucleotides or deoxyribonucleotides. This term refers only to the primary structure of the molecule. Thus, the term includes triple-, double- and single-stranded DNA, as well as triple-, double- and single-stranded RNA. It also includes modifications, such as by methylation and/or by capping, and unmodified forms of the polynucleotide. There is no intended distinction in length between the terms “nucleic acid,” “nucleic acid molecule,” “polynucleotide,” and “oligonucleotide” and these terms will be used interchangeably.

The terms “protein,” “polypeptide,” and “peptide” refer to any compound comprising naturally occurring or synthetic amino acid polymers or amino acid-like molecules including but not limited to compounds comprising amino and/or imino molecules. No particular size is implied by use of the terms “protein,” “polypeptide,” and “peptide,” and these terms are used interchangeably.

The term “tagging enzyme” refers to an enzyme that catalyzes a reaction which leads to the conjugation of a tag to a set of molecules, for example, nucleic acids, proteins, carbohydrates, or lipids. In some embodiments, a tagging enzyme catalyzes a reaction that results in promiscuous labeling of molecules, e.g., nucleic acids and/or proteins in the vicinity of the enzyme.

The term “tagging substrate” refers to a substrate of a tagging enzyme that, during the tagging enzyme-catalyzed reaction, is converted into a reactive form (e.g., a radical or unstable intermediate with a reactive functional group), which reacts with and attaches to a molecule (e.g., a nucleic acid or protein) in the vicinity of the enzyme. In some embodiments, a reactive moiety of the tagging substrate attaches to a molecule by formation of a covalent bond between the tagging substrate and the molecule.

As used herein, the term “binding pair” refers to first and second molecules that specifically bind to each other, such as a ligand and a receptor, an antigen and an antibody, or biotin and streptavidin. “Specific binding” of the first member of the binding pair to the second member of the binding pair in a sample is evidenced by the binding of the first member to the second member, or vice versa, with greater affinity and specificity than to other components in the sample. The binding between the members of the binding pair is typically noncovalent.

As used herein, a “solid support” refers to a solid surface such as a magnetic bead, latex bead, microtiter plate well, glass plate, nylon, agarose, acrylamide, and the like.

“Recombinant” as used herein to describe a nucleic acid molecule means a polynucleotide of genomic, cDNA, viral, semisynthetic, or synthetic origin which, by virtue of its origin or manipulation is not associated with all or a portion of the polynucleotide with which it is associated in nature. The term “recombinant” as used with respect to a protein or polypeptide means a polypeptide produced by expression of a recombinant polynucleotide. In general, the gene of interest is cloned and then expressed in transformed organisms, as described further below. The host organism expresses the foreign gene to produce the protein under expression conditions.

The terms “fusion protein,” “fusion polypeptide,” or “fusion peptide” as used herein refer to a fusion comprising a tagging enzyme in combination with a protein of interest as part of a single continuous chain of amino acids, which chain does not occur in nature. The tagging enzyme and the protein of interest may be connected directly to each other by peptide bonds or may be separated by intervening amino acid sequences. The protein of interest may be, for example, a cytosolic protein, a nuclear protein, a membrane protein, a mitochondrial protein, a P-body protein, a secretory pathway protein, or any other protein, wherein mapping its location and/or identifying it binding partners and/or nucleic acids in the vicinity of it in a cell is of interest. The fusion proteins may also contain other sequences such as targeting or localization sequences and/or tag sequences.

By “fragment” is intended a molecule consisting of only a part of the intact full length sequence and structure. The fragment can include a C-terminal deletion an N-terminal deletion, and/or an internal deletion of the polypeptide. Active fragments of a particular protein or polypeptide will generally include at least about 5-14 contiguous amino acid residues of the full-length molecule, but may include at least about 15-25 contiguous amino acid residues of the full-length molecule, and can include at least about 20-50 or more contiguous amino acid residues of the full-length molecule, or any integer between 5 amino acids and the full-length sequence, provided that the fragment in question retains biological activity.

“Substantially purified” generally refers to isolation of a substance (compound, polynucleotide, protein, polypeptide, peptide composition) such that the substance comprises the majority percent of the sample in which it resides. Typically, in a sample, a substantially purified component comprises 50%, preferably 80%-85%, more preferably 90-95% of the sample. Techniques for purifying polynucleotides and polypeptides of interest are well-known in the art and include, for example, ion-exchange chromatography, affinity chromatography and sedimentation according to density.

By “isolated” is meant, when referring to a protein, polypeptide or peptide, that the indicated molecule is separate and discrete from the whole organism with which the molecule is found in nature or is present in the substantial absence of other biological macro molecules of the same type. The term “isolated” with respect to a nucleic acid is a nucleic acid molecule devoid, in whole or part, of sequences normally associated with it in nature; or a sequence, as it exists in nature, but having heterologous sequences in association therewith; or a molecule disassociated from the chromosome.

The term “transformation” refers to the insertion of an exogenous polynucleotide into a host cell, irrespective of the method used for the insertion. For example, direct uptake, transduction or f-mating are included. The exogenous polynucleotide may be maintained as a non-integrated vector, for example, a plasmid, or alternatively, may be integrated into the host genome.

“Recombinant host cells,” “host cells,” “cells”, “cell lines,” “cell cultures,” and other such terms denoting microorganisms or higher eukaryotic cell lines cultured as unicellular entities refer to cells which can be, or have been, used as recipients for recombinant vector or other transferred DNA, and include the original progeny of the original cell which has been transfected.

A “coding sequence” or a sequence which “encodes” a selected polypeptide, is a nucleic acid molecule which is transcribed (in the case of DNA) and translated (in the case of mRNA) into a polypeptide in vivo when placed under the control of appropriate regulatory sequences (or “control elements”). The boundaries of the coding sequence can be determined by a start codon at the 5′ (amino) terminus and a translation stop codon at the 3′ (carboxy) terminus. A coding sequence can include, but is not limited to, cDNA from viral, prokaryotic or eukaryotic mRNA, genomic DNA sequences from viral or prokaryotic DNA, and even synthetic DNA sequences. A transcription termination sequence may be located 3′ to the coding sequence.

Typical “control elements,” include, but are not limited to, transcription promoters, transcription enhancer elements, transcription termination signals, polyadenylation sequences (located 3′ to the translation stop codon), sequences for optimization of initiation of translation (located 5′ to the coding sequence), and translation termination sequences.

“Operably linked” refers to an arrangement of elements wherein the components so described are configured so as to perform their usual function. Thus, a given promoter operably linked to a coding sequence is capable of effecting the expression of the coding sequence when the proper enzymes are present. The promoter need not be contiguous with the coding sequence, so long as it functions to direct the expression thereof. Thus, for example, intervening untranslated yet transcribed sequences can be present between the promoter sequence and the coding sequence and the promoter sequence can still be considered “operably linked” to the coding sequence.

“Encoded by” refers to a nucleic acid sequence which codes for a polypeptide sequence, wherein the polypeptide sequence or a portion thereof contains an amino acid sequence of at least 3 to 5 amino acids, more preferably at least 8 to 10 amino acids, and even more preferably at least 15 to 20 amino acids from a polypeptide encoded by the nucleic acid sequence.

“Expression cassette” or “expression construct” refers to an assembly which is capable of directing the expression of the sequence(s) or gene(s) of interest. An expression cassette generally includes control elements, as described above, such as a promoter which is operably linked to (so as to direct transcription of) the sequence(s) or gene(s) of interest, and often includes a polyadenylation sequence as well. Within certain embodiments of the invention, the expression cassette described herein may be contained within a plasmid construct. In addition to the components of the expression cassette, the plasmid construct may also include, one or more selectable markers, a signal which allows the plasmid construct to exist as single stranded DNA (e.g., a M13 origin of replication), at least one multiple cloning site, and a “mammalian” origin of replication (e.g., a SV40 or adenovirus origin of replication).

The term “transfection” is used to refer to the uptake of foreign DNA by a cell. A cell has been “transfected” when exogenous DNA has been introduced inside the cell membrane. A number of transfection techniques are generally known in the art. See, e.g., Graham et al. (1973) Virology, 52:456, Sambrook et al. (2001) Molecular Cloning, a laboratory manual, 3rd edition, Cold Spring Harbor Laboratories, New York, Davis et al. (1995) Basic Methods in Molecular Biology, 2nd edition, McGraw-Hill, and Chu et al. (1981) Gene 13:197. Such techniques can be used to introduce one or more exogenous DNA moieties into suitable host cells. The term refers to both stable and transient uptake of the genetic material, and includes uptake of peptide- or antibody-linked DNAs.

A “vector” is capable of transferring nucleic acid sequences to target cells (e.g., viral vectors, non-viral vectors, particulate carriers, and liposomes). Typically, “vector construct,” “expression vector,” and “gene transfer vector,” mean any nucleic acid construct capable of directing the expression of a nucleic acid of interest and which can transfer nucleic acid sequences to target cells. Thus, the term includes cloning and expression vehicles, as well as viral vectors.

“Gene transfer” or “gene delivery” refers to methods or systems for reliably inserting DNA or RNA of interest into a host cell. Such methods can result in transient expression of non-integrated transferred DNA, extrachromosomal replication and expression of transferred replicons (e.g., episomes), or integration of transferred genetic material into the genomic DNA of host cells. Gene delivery expression vectors include, but are not limited to, vectors derived from bacterial plasmid vectors, viral vectors, non-viral vectors, alphaviruses, pox viruses and vaccinia viruses.

### II. Modes of Carrying Out the Invention

Before describing the present invention in detail, it is to be understood that this invention is not limited to particular formulations or process parameters as such may, of course, vary. It is also to be understood that the terminology used herein is for the purpose of describing particular embodiments of the invention only, and is not intended to be limiting.

Although a number of methods and materials similar or equivalent to those described herein can be used in the practice of the present invention, the preferred materials and methods are described herein.

The present invention relates to the development of a novel method for determining the subcellular localization of nucleic acids. In particular, the method utilizes proximity-specific tagging of nucleic acids to identify nucleic acids, including DNA or RNA molecules in proximity to a protein of interest or within or near a particular subcellular compartment in vivo.

The method typically comprises the following steps: a) introducing a tagging enzyme into a cell, wherein the tagging enzyme is targeted to a subcellular region of interest; b) contacting the cell with a tagging substrate for the tagging enzyme, wherein the tagging enzyme catalyzes a reaction with the tagging substrate resulting in covalent attachment of a tag to nucleic acids within an intracellular spatial location around the tagging enzyme; and c) isolating the tagged nucleic acids using an agent that selectively binds to the tag; and c) analyzing the tagged nucleic acids to produce a map of the subcellular localization of the nucleic acids.

The method may be applied to cell samples comprising a single cell or a population of cells of interest and can be performed on any type of cell, including any cell from a prokaryotic, eukaryotic, or archaeon organism, including bacteria, archaea, fungi, protists, plants, and animals. Cells from tissues, organs, and biopsies, as well as recombinant cells, cells from cell lines cultured in vitro, and artificial cells (e.g., nanoparticles, liposomes, polymersomes, or microcapsules encapsulating nucleic acids) may all be used in the practice of the invention. The methods of the invention are also applicable for investigating nucleic acid localization in cellular fragments, cell components, or organelles comprising nucleic acids.

Although the methods for tagging and the related reagents, materials and compositions described herein are well suited for use in live cells and tissues, it should be appreciated that their use is not so limited, but that they can also be applied to fixed cells and tissues, for example, fixed cells and tissues obtained from a subject, e.g., in a clinical setting. The methods may also be applied to lysed cells.

In general, the methods and strategies for tagging nucleic acids employ a tagging enzyme. In some embodiments, the tagging enzyme catalyzes a reaction with a tagging substrate that generates a reactive unstable reagent (e.g., a radical or reaction intermediate with a reactive functional group) that is capable of covalently labeling nearby nucleic acids. The half-life of the tagging reagent generated by the tagging enzyme determines how far the reagent can travel from its point of generation before reacting with a molecule. Accordingly, the half-life of the reagent determines its labeling radius. Because the enzyme generated reagent has a short half-life, only nucleic acids in proximity to the tagging enzyme and the reactive reagent generated by the tagging enzyme (typically a few tens to hundreds of nanometers) are covalently modified (i.e., tagged).

The tagging enzyme can be introduced into a cell and contacted with a tagging substrate under conditions suitable for the tagging enzyme to convert the tagging substrate into a reactive form that can react with and attach to nucleic acids in the vicinity of the tagging enzyme. The tagging enzyme may be delivered to the cell interior or exterior, depending on which region of the cell is being analyzed. In some embodiments, the tagging enzyme is delivered to the interior of the cell, and in some instances, to specific subcellular compartments. In some embodiments, the tagging enzyme is delivered to a tissue. The tagging enzyme may also be introduced into a cell by transfecting the cell with a recombinant polynucleotide comprising a promoter operably linked to a polynucleotide encoding the tagging enzyme. The recombinant polynucleotide may comprise an expression vector, for example, a bacterial plasmid vector or a viral expression vector, such as, but not limited to, an adenovirus, retrovirus (e.g., γ-retrovirus and lentivirus), poxvirus, adeno-associated virus, baculovirus, or herpes simplex virus vector.

In some embodiments, the tagging enzyme is engineered to improve its capability in proximity labeling. For example, the tagging enzyme can be engineered to be expressed and/or active only within a subcellular compartment or structure of interest. The tagging enzyme may also be engineered to comprise one or more mutations that enhance its catalytic activity with a tagging substrate in a subcellular compartment or structure of interest.

The tagging enzyme can be directed to a specific protein or cellular compartment of interest in a number of ways. For example, the tagging enzyme may be modified to include a targeting sequence that directs the tagging enzyme to the subcellular region of interest. Targeting sequences that can be used include, but are not limited to, a secretory protein signal sequence, a membrane protein signal sequence, a nuclear localization sequence, a mitochondrial localization sequence, an outer mitochondrial membrane sequence, an endoplasmic reticulum localization sequence, an endoplasmic reticulum membrane targeting sequence, a nucleolar localization signal sequence, a nuclear export signal sequence, a peroxisome localization sequence, and a protein binding motif sequence. Exemplary targeting sequences are shown in Table 1 and include sequences selected from the group consisting of SEQ ID NOS:1-5.

In other embodiments, the tagging enzyme is covalently linked to a peptide or protein that directs the tagging enzyme to a subcellular region of interest, such as a cytosolic protein, a nuclear protein, a membrane protein, a mitochondrial protein, a P-body protein, or a secretory pathway protein. Attachment of the tagging enzyme to the protein of interest results in proximity labeling of nucleic acids surrounding the protein of interest in the locations where the protein resides in the cell. Alternatively, the tagging enzyme can be covalently linked to an antibody that specifically binds to a particular epitope found on certain proteins in a subcellular region of interest, which similarly allows proximity labeling of surrounding nearby nucleic acids.

In some embodiments, the tagging enzyme is a peroxidase. Peroxidases catalyze the reaction of phenol and phenolic compounds such as tyramine or phenolic aryl azide derivatives with hydrogen peroxide to generate short-lived, reactive free radicals. For example, proximity labeling can be performed in the presence of hydrogen peroxide and biotin-phenol (BP) or a derivative thereof (e.g., O-acetylated biotin-phenol), wherein the peroxidase catalyzes the reaction of the biotin-phenol with the hydrogen peroxide to produce a biotin-phenoxyl radical that reacts with nearby nucleic acids resulting in biotinylation (i.e., tagging) of the nucleic acids. Exemplary peroxidases suitable for use as tagging enzymes include horseradish peroxidase, soybean peroxidase, and ascorbate peroxidase. In certain embodiments, the tagging enzyme is an engineered ascorbate peroxidase (e.g., APEX or APEX2). An advantage of using certain engineered ascorbate peroxidases is they can be expressed and active in a reducing cellular environment. For a description of APEX and APEX2 engineered ascorbate peroxidases, see. e.g., Martell et al. (2012) Nat. Biotechnol. 30:1143-1148, Lam et al. (2015) Nat. Methods 12:51-54, and U.S. Patent Application Publication No. U.S. 2014/0186870; herein incorporated by reference in their entireties.

Biotinylated nucleic acids, produced as described herein, can be isolated with a biotin-binding protein, such as streptavidin or avidin. The biotin-binding protein may be immobilized on a solid support (e.g., streptavidin beads or magnetic beads) to facilitate removal from a liquid. The isolated nucleic acids can then be analyzed to identify the DNA and/or RNA molecules by any appropriate method (e.g., sequencing or polymerase chain reaction (PCR) with suitable primers for identification of the nucleic acids). RNA may be reverse transcribed into cDNA with a reverse transcriptase prior to performing PCR (i.e., RT-PCR) and/or sequencing.

Any high-throughput technique for sequencing the nucleic acids can be used in the practice of the invention. Deep sequencing of nucleic acids can be used, for example, to improve sequence accuracy and for determining the frequency of RNA molecules in particular subcellular compartments or regions. DNA sequencing techniques include dideoxy sequencing reactions (Sanger method) using labeled terminators or primers and gel separation in slab or capillary, sequencing by synthesis using reversibly terminated labeled nucleotides, pyrosequencing, 454 sequencing, sequencing by synthesis using allele specific hybridization to a library of labeled clones followed by ligation, real time monitoring of the incorporation of labeled nucleotides during a polymerization step, polony sequencing, SOLID sequencing, and the like.

Certain high-throughput methods of sequencing comprise a step in which individual molecules are spatially isolated on a solid surface where they are sequenced in parallel. Such solid surfaces may include nonporous surfaces (such as in Solexa sequencing, e.g. Bentley et al, Nature, 456: 53-59 (2008) or Complete Genomics sequencing, e.g. Drmanac et al, Science, 327: 78-81 (2010)), arrays of wells, which may include bead- or particle-bound templates (such as with 454, e.g. Margulies et al, Nature, 437: 376-380 (2005) or Ion Torrent sequencing, U.S. patent publication 2010/0137143 or 2010/0304982), micromachined membranes (such as with SMRT sequencing, e.g. Eid et al, Science, 323: 133-138 (2009)), or bead arrays (as with SOLiD sequencing or polony sequencing, e.g. Kim et al, Science, 316: 1481-1414 (2007)). Such methods may comprise amplifying the isolated molecules either before or after they are spatially isolated on a solid surface. Prior amplification may comprise emulsion-based amplification, such as emulsion PCR, or rolling circle amplification.

Of particular interest is sequencing on the Illumina MiSeq, NextSeq, and HiSeq platforms, which use reversible-terminator sequencing by synthesis technology (see, e.g., Shen et al. (2012) BMC Bioinformatics 13:160; Junemann et al. (2013) Nat. Biotechnol. 31(4):294-296; Glenn (2011) Mol. Ecol. Resour. 11(5):759-769; Thudi et al. (2012) Brief Funct. Genomics 11(1):3-11; herein incorporated by reference).

As discussed above, tagging enzymes can be genetically targeted to a cellular region of interest to identify nucleic acids in a specific subcellular compartment or region (e.g., the nucleus, endoplasmic reticulum, Golgi, mitochondria, mitochondria outer membrane, mitochondria inner membrane, mitochondria matrix space, chloroplasts, synaptic cleft, presynaptic membrane, postsynaptic membrane, dendritic spines, transport vesicles, regions of contact between mitochondria and endoplasmic reticulum, nuclear membrane, etc.).

In some embodiments, the tagging enzyme is fused to a protein of interest that localizes within particular cell types (e.g., astrocytes, dendrocytes, immune cells, stem cells, etc.) to allow cellular nucleic acids in the vicinity of the protein of interest to be specifically tagged. For example, the protein of interest may localize within a specific cell type within a complex tissue, animal, or cell population. In some embodiments, the protein of interest forms particular macromolecular complexes (e.g., protein complexes such as ribosomes, replisome, transcription complex, spliceosome, DNA repair complex, fatty acid synthase, polyketide synthase, non-ribosomal peptide synthase, glutamate receptor signaling complex, neurexin-neuroligin signaling complex, etc.). In each context, the tagged nucleic acids in the vicinity of the protein of interest can be analyzed (e.g., isolated and identified) to map protein-nucleic acid localization for specific cells, cellular compartments or regions, or macromolecular complexes of interest. This information can be used for research, diagnostic, therapeutic, and other applications.

For example, cells may be isolated from a patient, amplified or differentiated using IPS cell technology (induced pluripotent stem cell), contacted with a vector (e.g., a viral vector) that expresses a tagging enzyme, for example, a tagging enzyme fused to a localization signal effecting localization of the tagging enzyme in a specific subcellular compartment. Labeling can be performed in the living cells, as described herein, and the resulting tagged nucleic acids can be analyzed, for example, to identify patient specific information that can be useful to assist in diagnostic, prognostic, and/or therapeutic decisions, and in drug screening assays.

A tagging substrate is typically provided in an inert, stable, or non-reactive form, e.g., a form that does not readily react with other molecules in living cells. Once in contact with an active tagging enzyme, the tagging substrate is converted from its stable form into a short-lived reactive form, for e.g., via generation of a reactive moiety, such as a radical, on the tagging substrate by the tagging enzyme. Some tagging substrates are, accordingly, also referred to as radical precursors. The reactive form of the tagging substrate then reacts with and attaches to a molecule, e.g., a nucleic acid, in the vicinity of the tagging enzyme. Accordingly, in some embodiments, a tagging substrate comprises an inert or stable moiety that can be converted by the tagging enzyme into a reactive moiety. The reaction of the tagging substrate with a molecule, e.g., a nucleic acid in the vicinity of the tagging enzyme, results in the tagging, or labeling, of the molecule. Typically, a tagging substrate comprises a tag, which is a functional moiety or structure that can be used to detect, identify, or isolate a molecule comprising the tag, e.g., a protein that has been tagged by reacting with a tagging substrate. Suitable tags include, but are not limited to, for example, a detectable label, a binding agent, such as biotin, or a fluorescent probe, a click chemistry handle, an azide, alkyne, phosphine, trans-cyclooctene, or a tetrazine moiety. In some embodiments, the reaction of the reactive form of the tagging substrate with a molecule, e.g., a nucleic acid, may lead to changes in the molecule, e.g., oxygenation, that can be exploited for detecting and/or isolating the changed molecules. Non-limiting examples of such tagging substrates are chromophores, e.g., resorufin, malachite green, KillerRed, Ru(bpy)₃²⁺, and miniSOG, which can generate reactive oxygen species that oxidize molecules in the vicinity of the respective tagging enzyme. The oxidation can be used to isolate and/or identify the oxidized molecules. In some embodiments, the reactive form of the tagging substrate crosses cell membranes, while in other embodiments membranes are impermeable to the reactive form of the tagging substrate.

A tag may be, in some embodiments, a detectable label. In some embodiments, a tag may be a functional moiety or structure that can be used to detect, isolate, or identify molecules comprising the tag. A tag may also be created as a result of a reactive form of a tagging substrate reacting with a molecule, e.g., the creation of oxidative damage by a reactive oxygen species may be a tag. In some embodiments, the tag is a biotin-based tag and the tagging enzyme, e.g., a peroxidase, generates a reactive biotin moiety that binds to nucleic acids within the vicinity of the tagging enzyme. In some embodiments, the biotin-based tags are biotin-phenol or tyramide molecules. In some embodiments, the tagging substrate is a peroxidase substrate.

Additional suitable tagging substrates will be apparent to those of skill in the art, and the invention is not limited in this respect. In some embodiments, the tag is an alkyne phenol or tyramide and the peroxidase generates a reactive moiety that binds to nucleic acids within the vicinity of the peroxidase. The alkyne subsequently can be modified, for example, by a click chemistry reaction to attach a tag (e.g., a biotin tag). The tag can then be used for further analysis (e.g., isolation and identification). It should be noted that the invention is not limited to alkyne phenol or tyramide, but that any functional group that can be chemoselectively derivatized can be used. Some examples are: azide or alkyne or phosphine, or trans-cyclooctene, or tetrazine, or cyclooctyne, or ketone, or hydrazide, or aldehyde, or hydrazine.

In some embodiments, a tagging substrate for a peroxidase, for example, a biotinylated phenol or tyramide, is administered to cells or tissue in vivo, and nucleic acids that are located within the vicinity of the expressed peroxidase are tagged, i.e., the biotin phenol or tyramide is converted into a reactive form by the tagging enzyme. Here the peroxidase, and the reactive form reacts with and attaches to nucleic acids in the vicinity of the peroxidase, resulting in biotin-tagging of the respective nucleic acids. In the presence of peroxide (e.g., H₂O₂), the peroxidase converts the substrate into a short-lived, reactive intermediate, for example, a reactive phenol or tyramide radical, that can form a covalent bond with a nucleic acid.

In some embodiments, the reactive intermediate, once created, reacts with (i.e., labels) nucleic acids that are within the vicinity of the peroxidase enzyme. The term “within the vicinity” refers to the spatial location around the enzyme and/or substrate that is labeled. In some instances, it may refer to a region of the cell such as a sub-cellular region, a membrane or protein complex. Alternatively, it can be defined in terms of distance from the enzyme or substrate or a region i.e., as a diameter, circumference or linear distance. For example, in some embodiments, a molecule within the vicinity of a tagging enzyme is a molecule that is positioned less than about 900 nm, less than about 800 nm, less than about 700 nm, less than about 600 nm, less than about 500 nm, less than about 400 nm, less than about 300 nm, less than about 200 nm, less than about 100 nm, less than about 90 nm, less than about 80 nm, less than about 70 nm, less than about 60 nm, less than about 50 nm, less than about 40 nm, less than about 30 nm, less than about 20 nm, or less than about 10 nm away from the active site of the tagging enzyme. In some embodiments, nucleic acids that are not within the vicinity of the enzyme are not exposed to the reactive intermediate and hence not labeled. In some embodiments, expression or targeting of the tagging enzyme to a subcellular compartment results in quantitative tagging of virtually all nucleic acids within that compartment.

In some embodiments, in vivo nucleic acid tagging is performed with a tagging enzyme that can be genetically targeted to any part of a live cell. In some embodiments, the tagging enzyme is present and/or active in all regions of the cell. In some embodiments, the tagging enzyme is present and/or active only in a subcellular compartment of the cell. In some embodiments, the tagging substrate is an exogenous small-molecule substrate that can be added or uncaged for the desired window of time, to permit precise temporal control of labeling. In some embodiments, the tagging substrate is conjugated to a binding agent, e.g., biotin (or other purification handle), for subsequent capture, e.g., by streptavidin-coated beads. In some embodiments, the tagging enzyme converts the substrate into a highly reactive species that has the potential to label any endogenous nucleic acid, in order to achieve high depth-of-coverage (e.g., deep sequencing). In some embodiments, the reactive species has a short half-life on that its diffusion radius before quenching is less than approximately 100 nm, to ensure high specificity. In some embodiments, it is preferable for the reactive species not to cross cell membranes, to allow mapping of membrane-bounded structures.

In some embodiments, a tagging enzyme is engineered to be expressed and/or targeted in vivo or in situ to specific cells, cellular compartments (e.g., endoplasmic reticulum, Golgi apparatus, mitochondria, nucleus, the synaptic cleft, transport vesicles, etc.), and/or macromolecular complexes (e.g., protein complexes such as ribosomes, nuclear pore complex, fatty acid synthases) of interest. In some embodiments, a tagging enzyme is engineered to tag nucleic acids that are located within a limited distance of the tagging enzyme. As a result, in some embodiments, nucleic acids that are located within the targeted cell, cellular compartment, and/or macromolecular complex (e.g., ribonucleoprotein complex) are specifically tagged relative to other nucleic acids that are not located near the tagging enzyme. It should be appreciated that the tagging process itself does not need to be specific. For example, in some embodiments, it is the specific localization of the tagging enzyme that results in the specific tagging of a subset of nucleic acids of interest. In some embodiments, nucleic acids that are present within the vicinity of the tagging enzyme may be tagged for further analysis. In some embodiments, all nucleic acids present within the vicinity of the tagging enzyme may be tagged. Various versions of the methodology offer a range of labeling radii, from about 500 nm to less than 10 nm, e.g., tagging radii of about 500 nm, about 400 nm, about 300 nm, about 250 nm, about 200 nm, about 100 nm, about 90 nm, about 80 nm, about 70 nm, about 60 nm, about 50 nm, about 40 nm, about 30 nm, about 20 nm, about 10 nm, about 5 nm, about 2.5 nm, or about 1 nm.

In some embodiments, the reactive moiety produced by the tagging enzyme, e.g., the peroxidase, can be inactivated by contacting it with a quenching agent (e.g., a radical quencher such as ascorbate, 6-hydroxy-2,5,7,8-tetramethylchroman-2-carboxylic acid (TROLOX), or sodium azide after tagging with a peroxidase). As a result, the reactive moiety can have a short half-life and only modify nucleic acids that are located within a short distance of the site of production (the peroxidase) before being inactivated. Accordingly, the zone of tagging can be limited by the diffusion rate of the reactive form of the tagging substrate, or the activated tagging moiety, and the half-life of the reactive form of the tagging substrate, or the activated tagging moiety.

In some embodiments, only nucleic acids that are located within about 10 nm of the tagging enzyme are tagged. For example, in some embodiments using a peroxidase and a biotinylated peroxidase tagging substrate, e.g., a biotinylated phenol or tyramide, only nucleic acids that are located within about 10 nm of the peroxidase are biotinylated. However, it should be appreciated that the zone of biotinylation may be altered depending on the enzyme and/or substrate structure used for tagging. Thus, the labeling range can be adjusted from about 500 nm to <10 nm.

The methods provided herein can also be used to map nucleic acid localization in specific cell types within complex tissues or heterogeneous cell populations, or of specific subcellular structures or organelles within specific cells in complex tissues or populations. The methods are particularly useful for mapping subcellular localization of nucleic acids in rare cells within complex cell populations.

Maps of subcellular localization of nucleic acids can be developed not only for different cells, subcellular compartments, tissues, or organisms but also for cells, tissues, or organisms exposed to different conditions or environments. For example, cells or organisms exposed to different therapeutic agents, different concentrations of therapeutic agents, and/or combinations of therapeutic agents may be mapped and analyzed independently or compared against one another to examine changes occurring within a cell, tissue, or organism. Additionally, changes in nucleic acid localization in cells, tissues, or organisms over time associated with diseased states can be monitored by comparison of mapped nucleic acid localization in cells, tissues, or organisms in diseased and normal (i.e. healthy control, not having the disease) states.

In certain embodiments, a map of the subcellular localization of nucleic acids molecules, produced by the methods described herein, is compared to a reference map. For example, a map of the subcellular localization of the RNA molecules from a cell that is exposed to a test condition can be compared to a reference map of a cell that is not exposed to the test condition. A test condition may comprise, for example, exposing the cell to a drug, a ligand for a receptor, a hormone, a second messenger, a pathogen, or a genetic modification. For example, the cell can be genetically modified by introducing a vector, short hairpin RNA (shRNA), small interfering RNA (siRNA), microRNA (miRNA), or CRISPR-associated system into the cell. Alternatively, a test condition may comprise exposing the cell to a change in temperature, growth media, membrane potential, or osmotic pressure. In certain embodiments, the cell is exposed to the test condition prior to said contacting the cell with the tagging substrate.

Maps of subcellular localization of nucleic acids can also be developed for cells, subcellular compartments, tissues, or organisms at different developmental stages. For example, a map of the subcellular localization of nucleic acids can be compared to reference maps for cells, subcellular compartments, tissues, or organisms at the same or different developmental stages.

### III. Experimental

Below are examples of specific embodiments for carrying out the present invention. The examples are offered for illustrative purposes only, and are not intended to limit the scope of the present invention in any way.

Efforts have been made to ensure accuracy with respect to numbers used (e.g., amounts, temperatures, etc.), but some experimental error and deviation should, of course, be allowed for.

### Example 1

### General Overview of the Method for Mapping the Spatial Localization of Cellular Nucleic Acids by Proximity-Dependent Enzymatic Tagging

Our method combines proximity-specific RNA biotinylation with RNA deep sequencing (RNA Seq), to identify RNAs within or near a particular subcellular compartment in vivo. First, transgenic cell lines or organisms are generated in which an enzyme capable of proximity-specific biotinylation of nucleic acids (such as but not restricted to horseradish peroxidase and ascorbate peroxidase and their mutants and derivatives) is targeted to the compartment of interest. Cells or organisms are then briefly treated with this enzyme's substrate(s), inducing pervasive biotinylation of nucleic acids within the target compartment. Immediately thereafter, cells are lysed, biotinylated nucleic acids are enriched by conventional methods, and analyzed by deep sequencing.

The method presented here uses standard genetic manipulation techniques and either commercially available or readily synthesizable reagents to provide an exquisitely sensitive, broad and unbiased view of subcellular RNA localization. Importantly, our proposed technique is not limited to mapping of mRNAs, i.e., RNAs that are bound to ribosomes; rather, we have shown that multiple classes of non-coding RNAs may also be enriched and mapped by our methodology.

Since all biological processes-including development and disease-fundamentally depend on both RNA function and cellular organization, we anticipate that this technology will enable a vast array of insights with potential clinical relevance. Identifying RNA mislocalization events that contribute to a diseased state may help in identifying new targets for therapeutic development. Likewise, comparing subcellular transcriptomes and proteomes may facilitate the identification of novel ribonucleoprotein (RNP) interactions, which may likewise be therapeutic targets. In a broader sense, characterization of the contributing factors (sequences, structures, binding partners, etc. . . . ) that specify RNA subcellular targeting may allow one to manipulate the localization of endogenous or artificial RNPs, a new avenue for the design of advanced RNA therapeutics.

### Example 2

### Engineered Ascorbate Peroxidase for Proximity Labeling of Nucleic Acids

Engineered ascorbate peroxidase (APEX) can be used for proximity labeling of nucleic acids. With exposure to hydrogen peroxide and biotin-phenol (BP), APEX generates a phenoxyl radical, which covalently reacts with proteins and nucleic acids in its vicinity. Biotin-labeled nucleic acids can be isolated by binding to streptavidin and identified by sequencing. Major advantages of APEX are that 1) APEX, unlike horseradish peroxidase (HRP), can be expressed and active in reducing environments and 2) the generated phenoxyl radical is reactive with nearby biomolecules before it diffuses away from APEX.

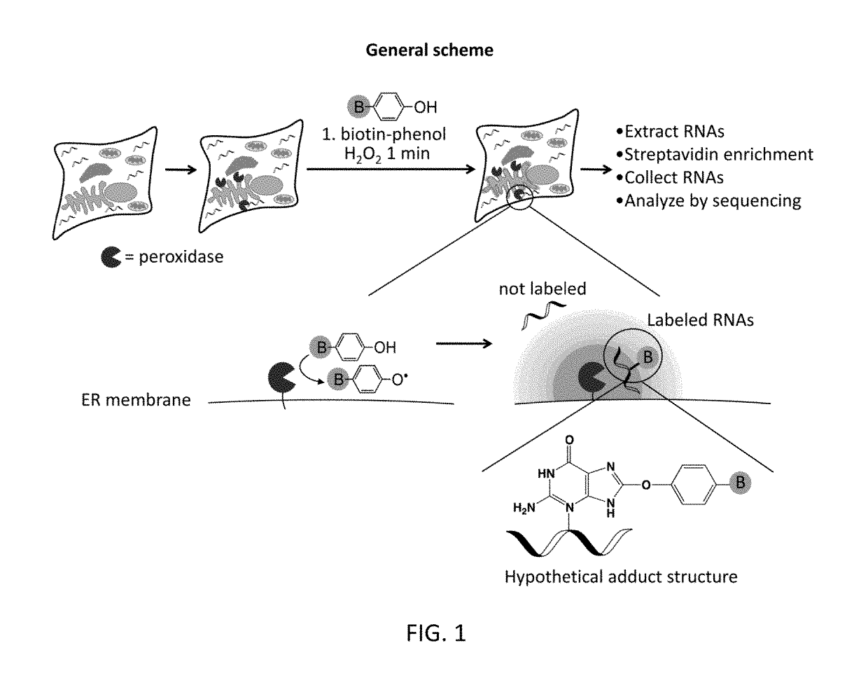

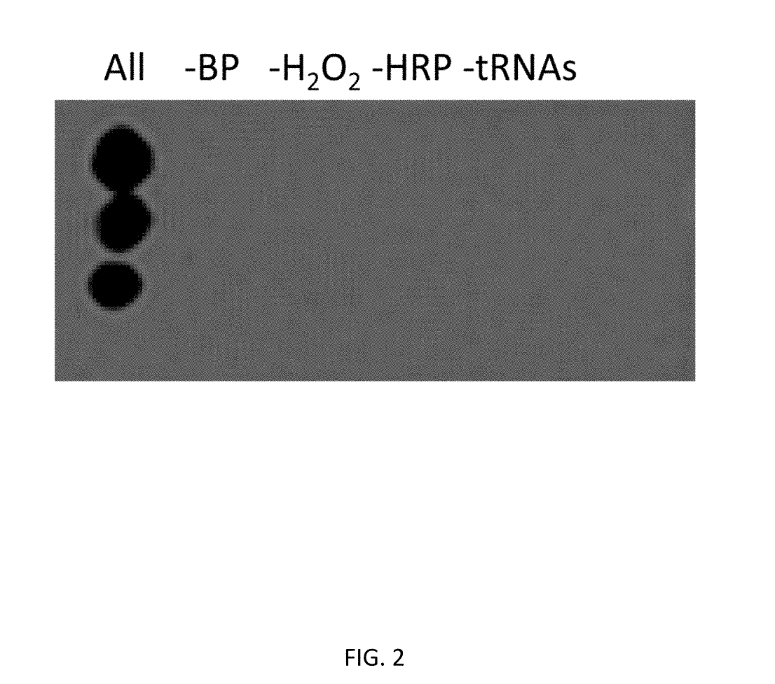

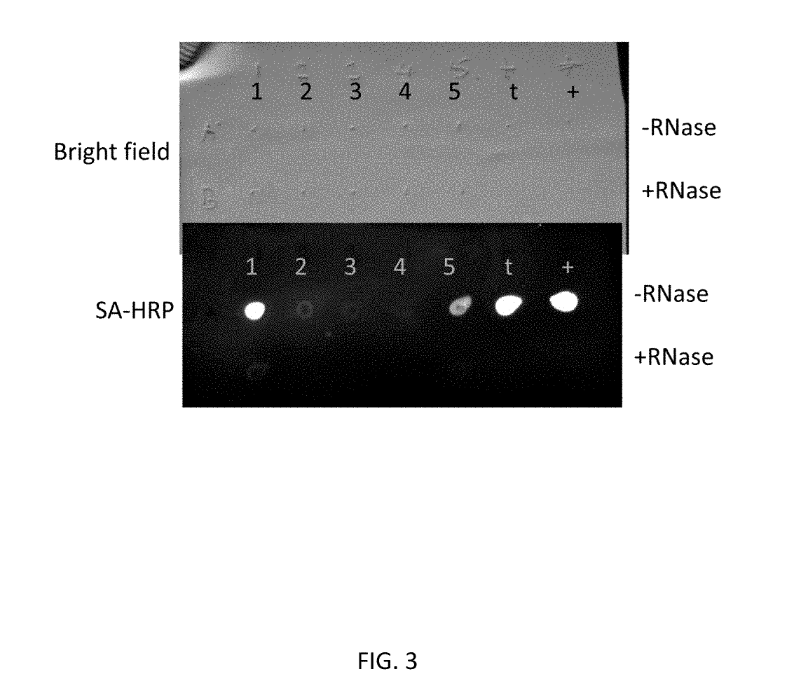

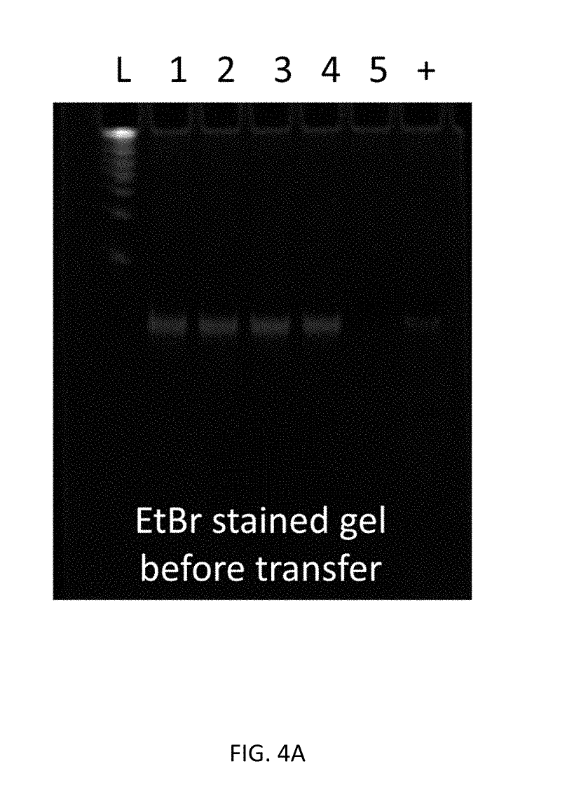

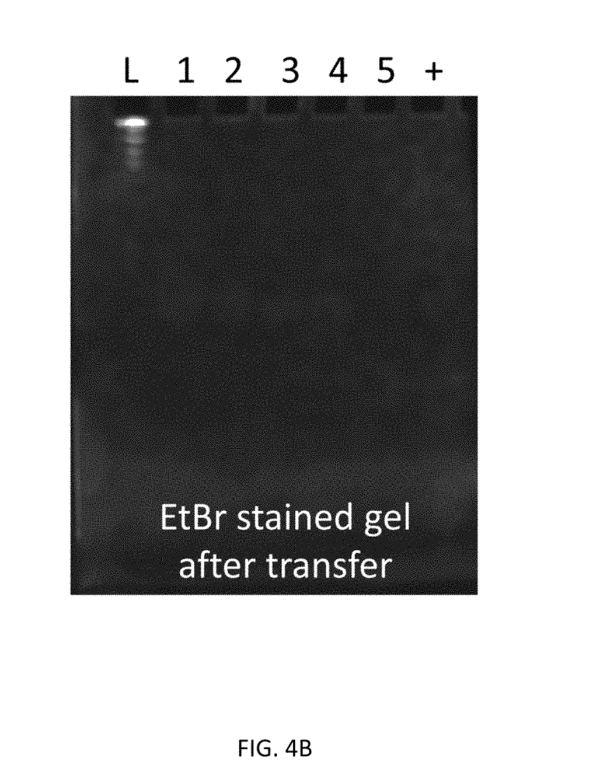

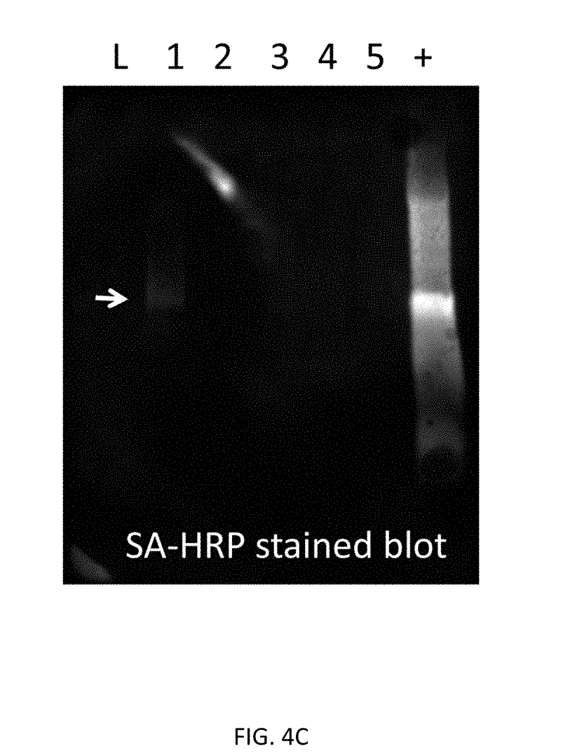

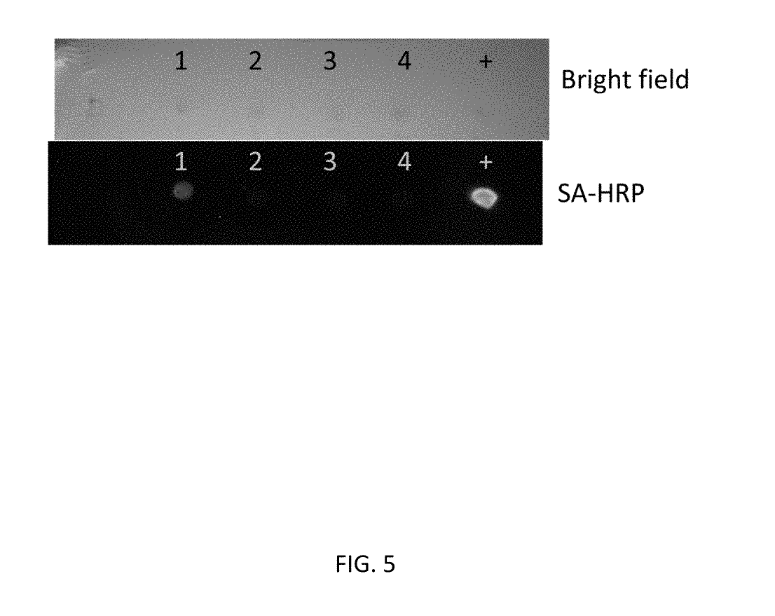

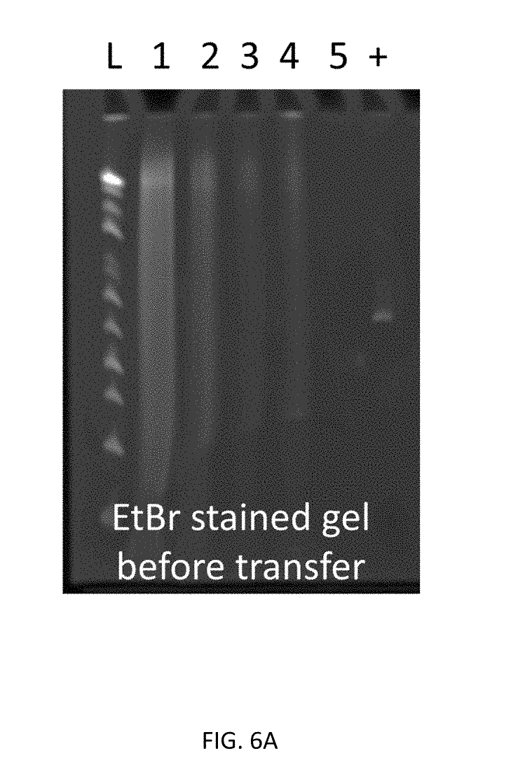

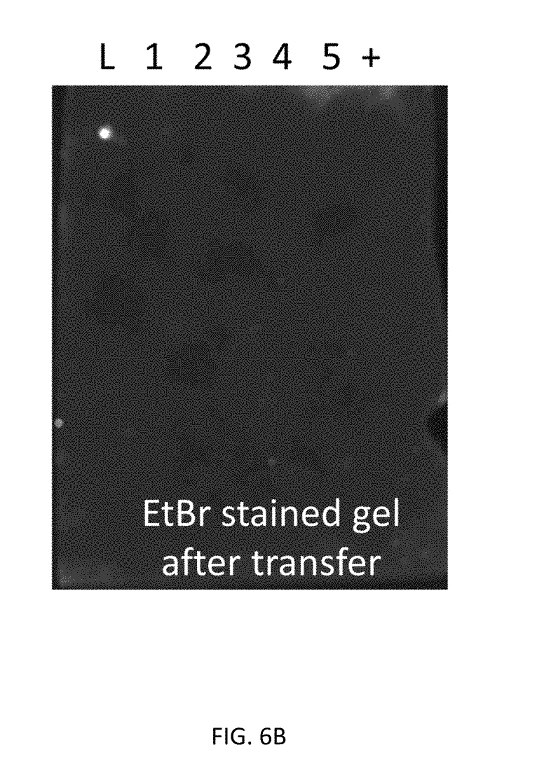

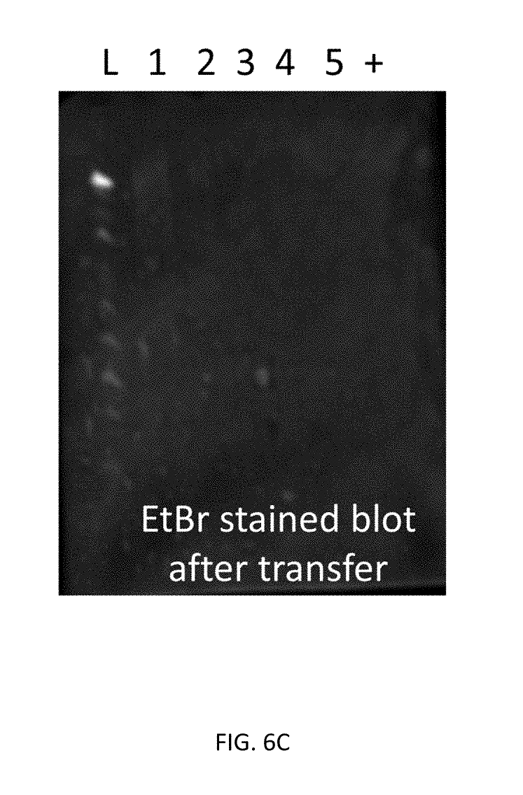

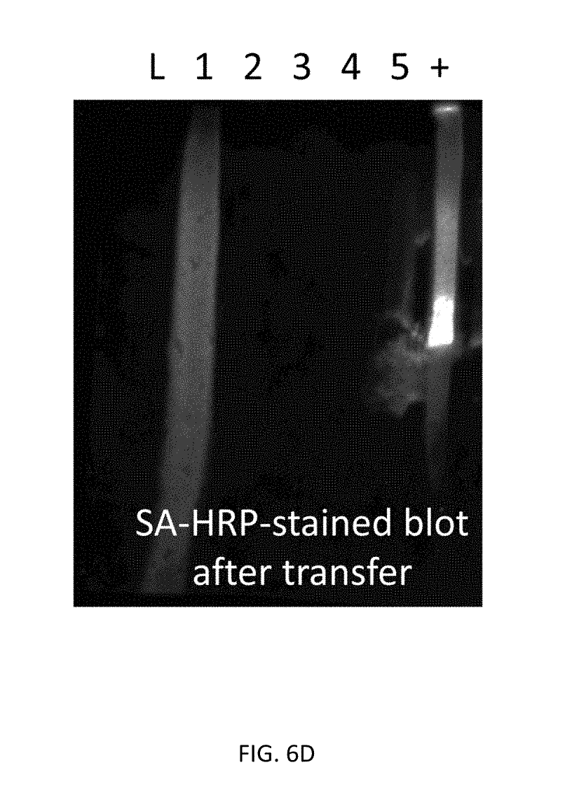

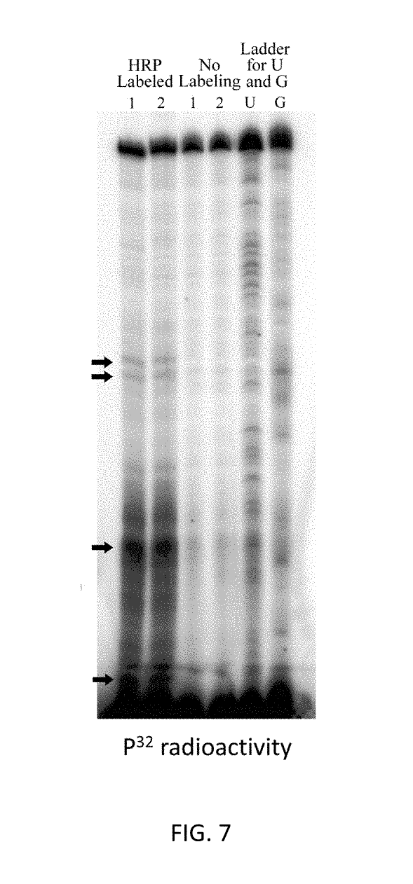

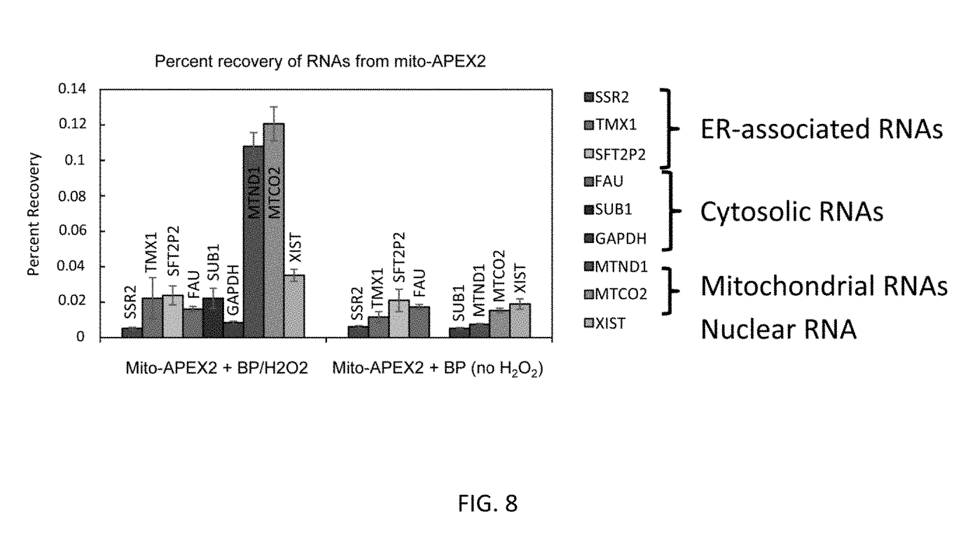

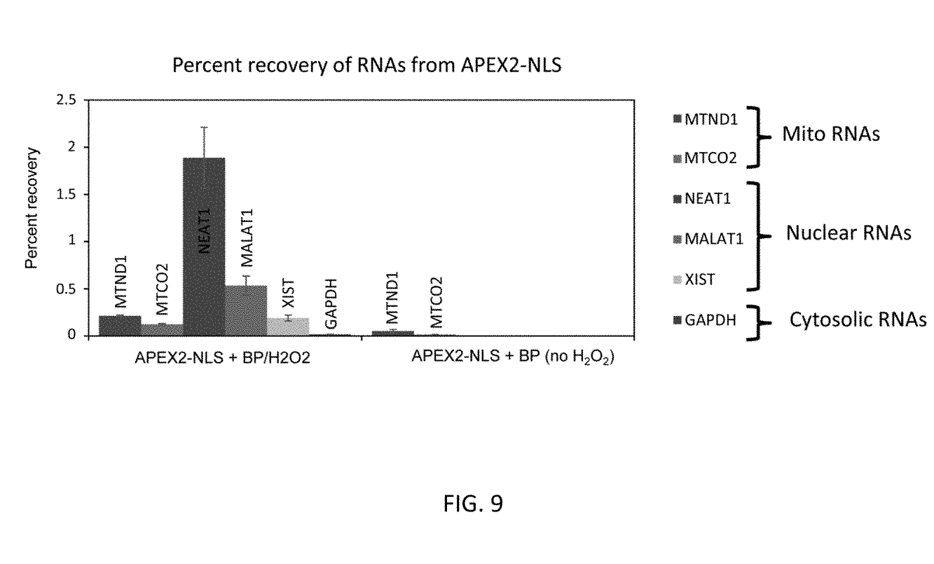

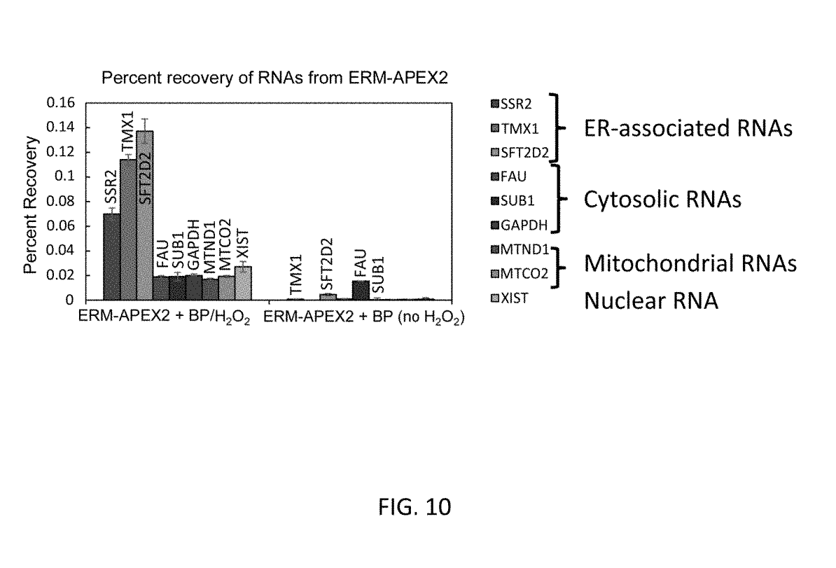

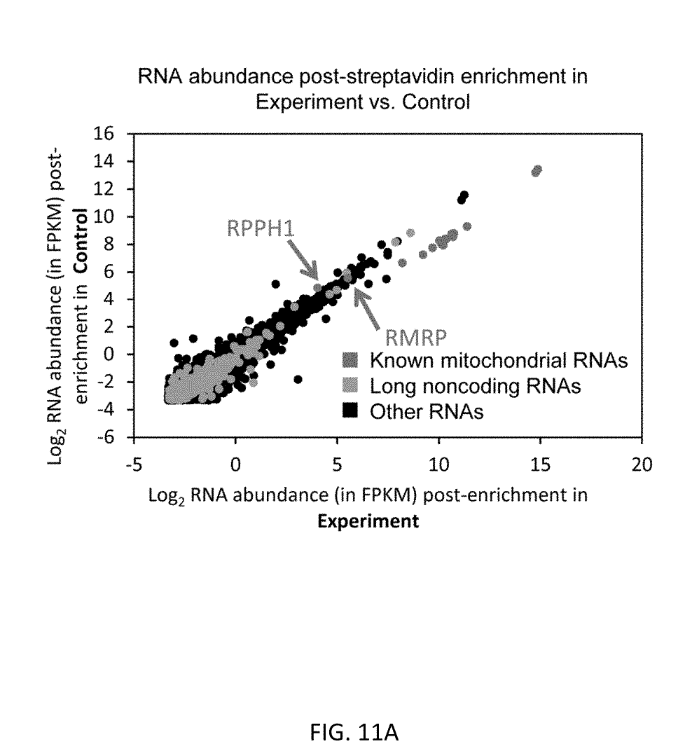

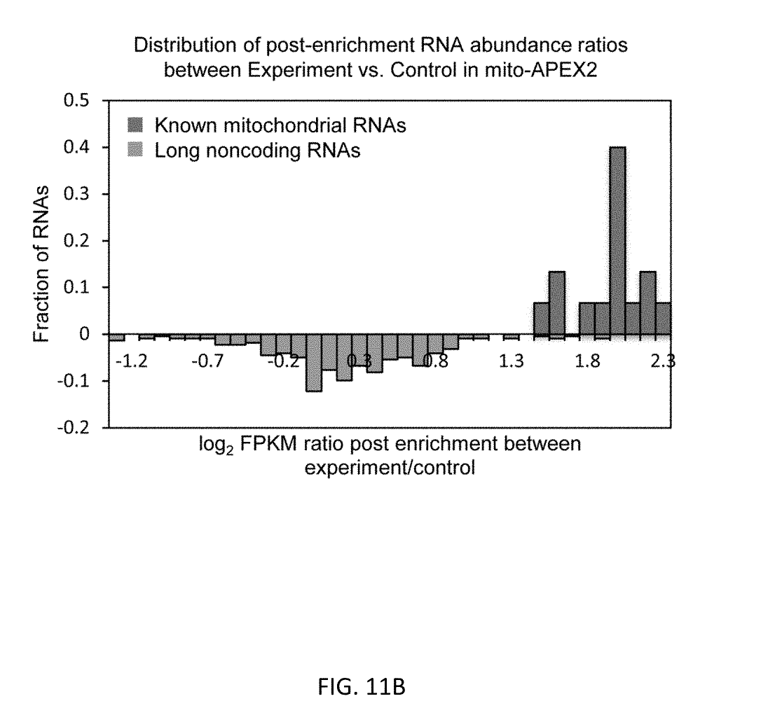

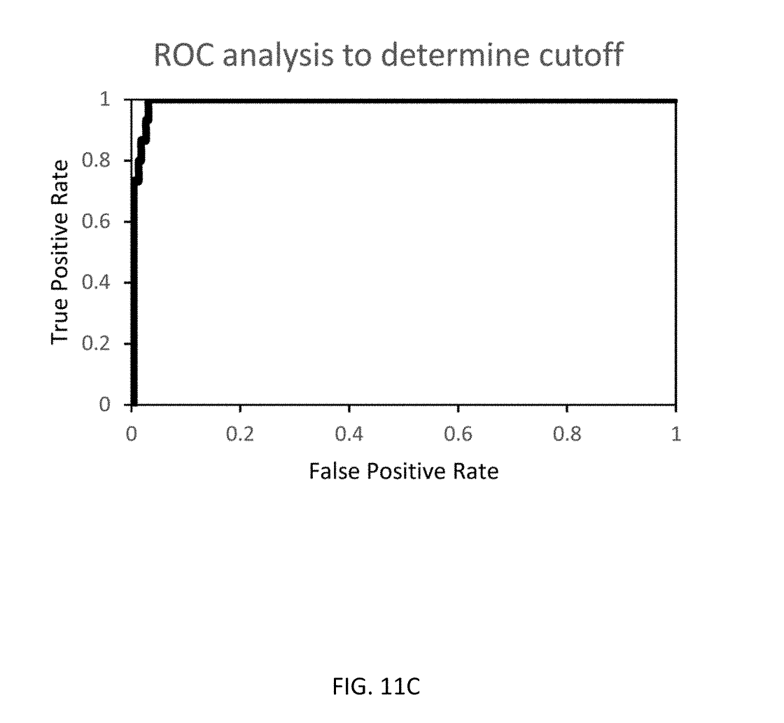

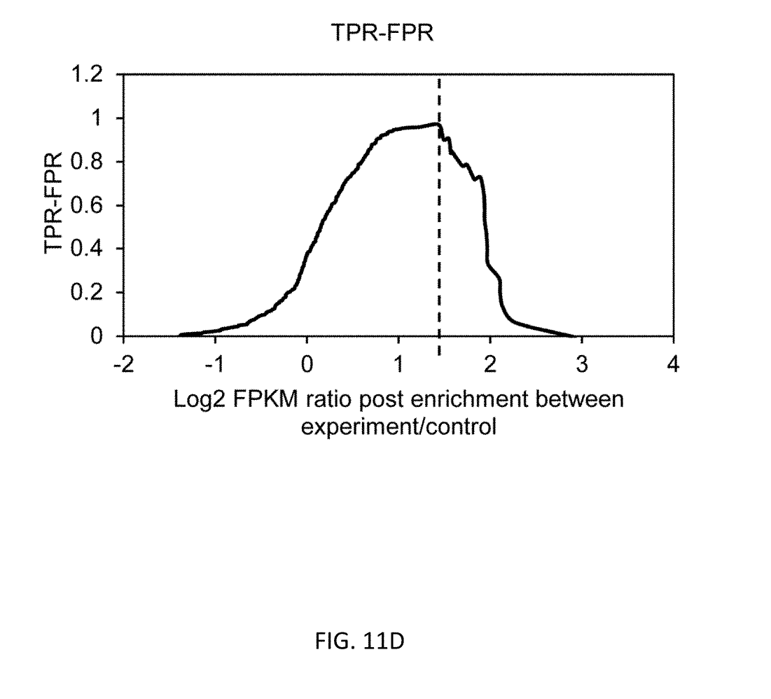

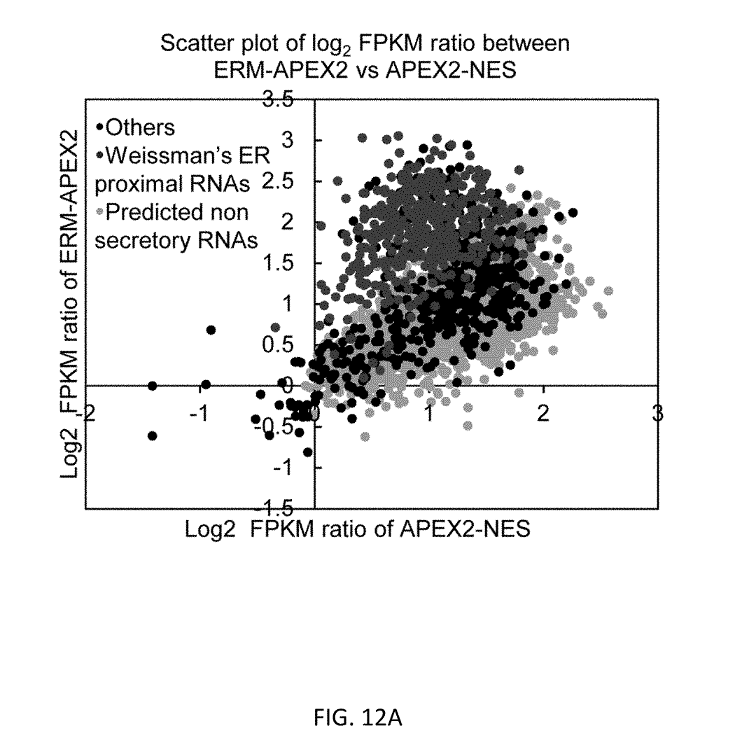

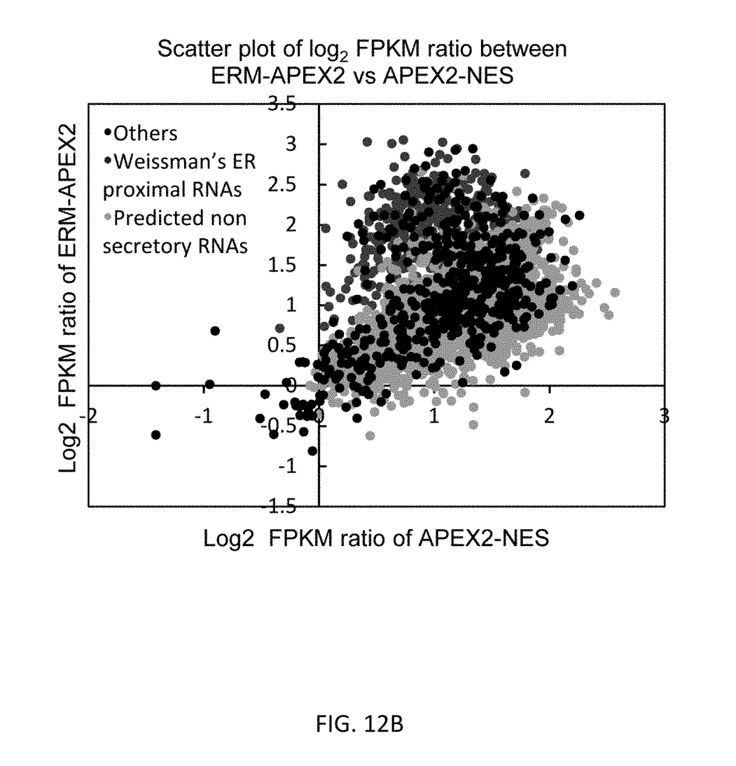

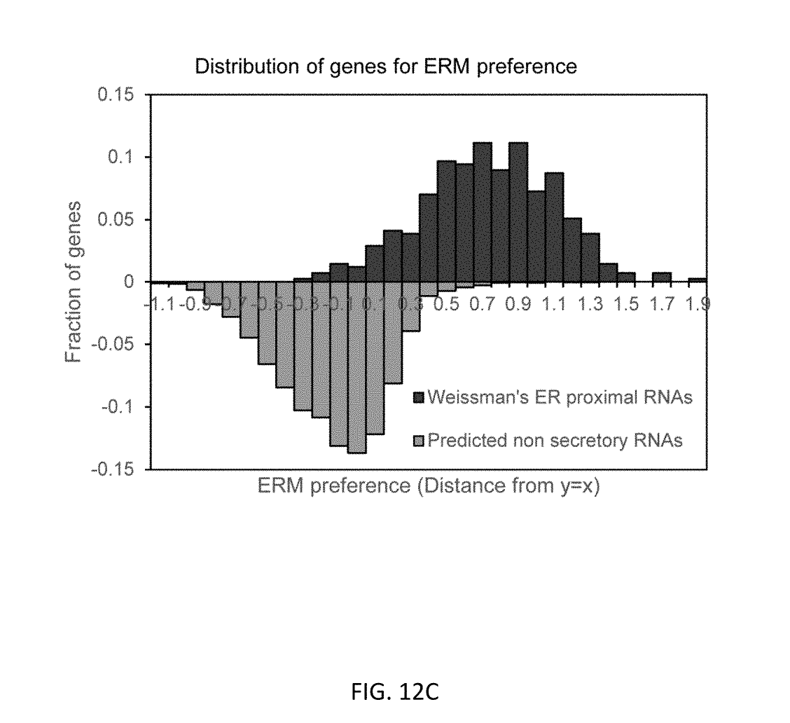

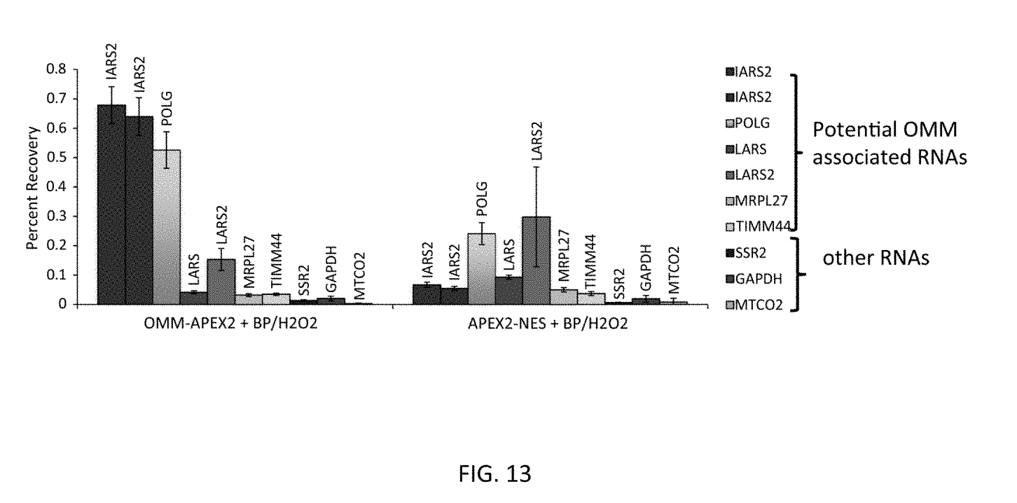

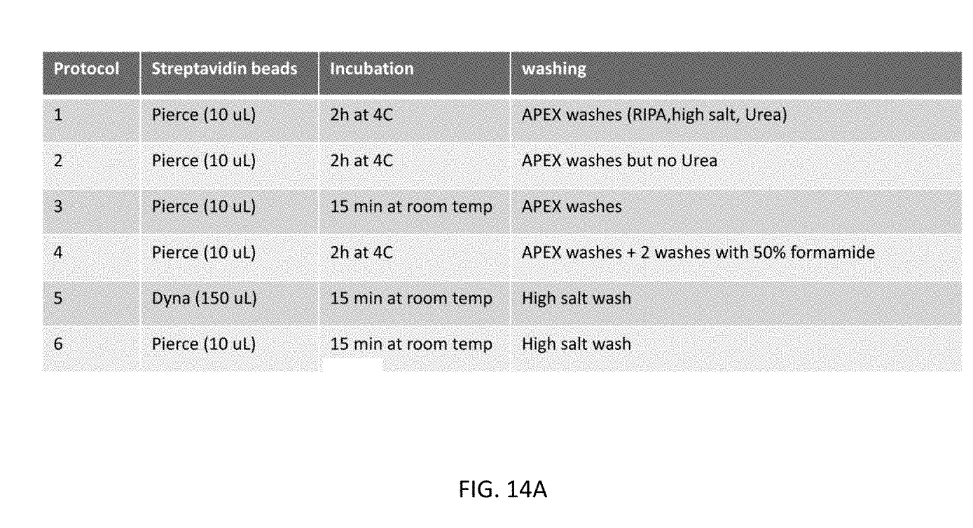

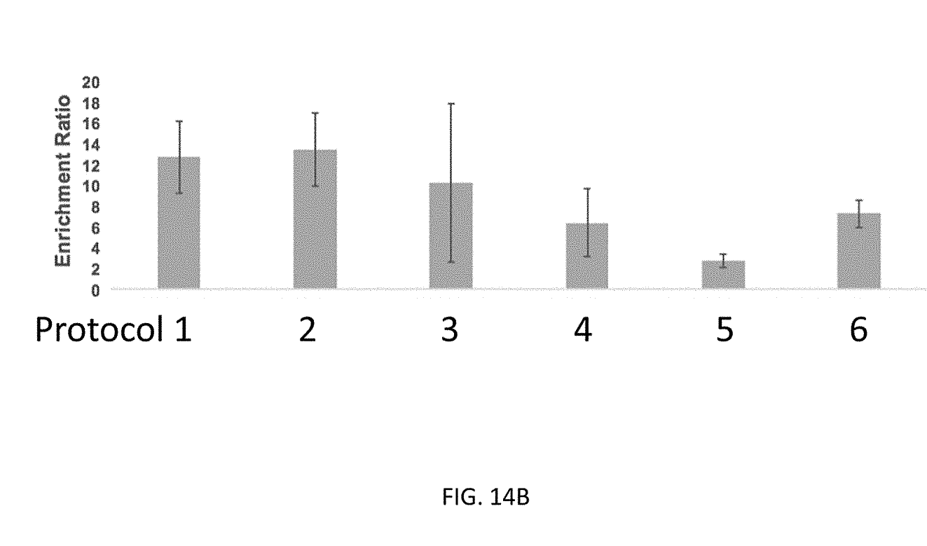

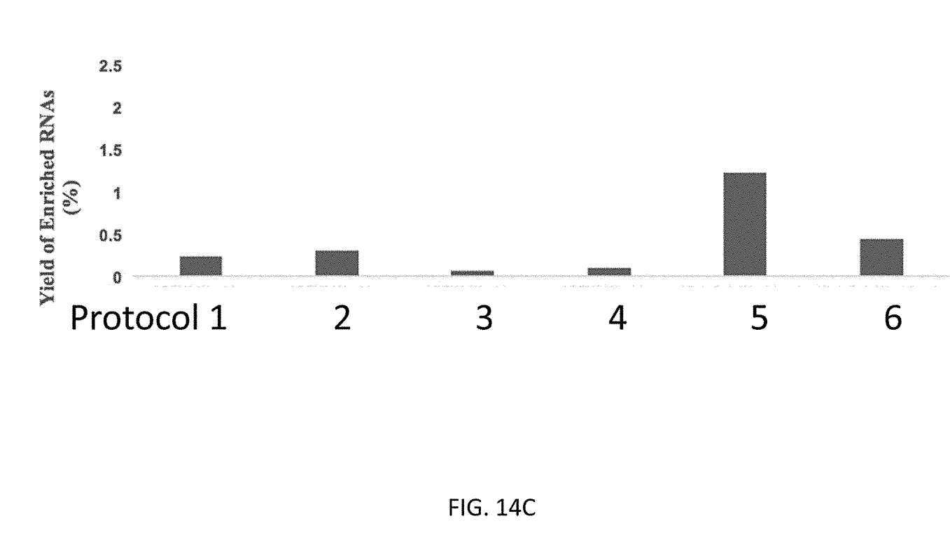

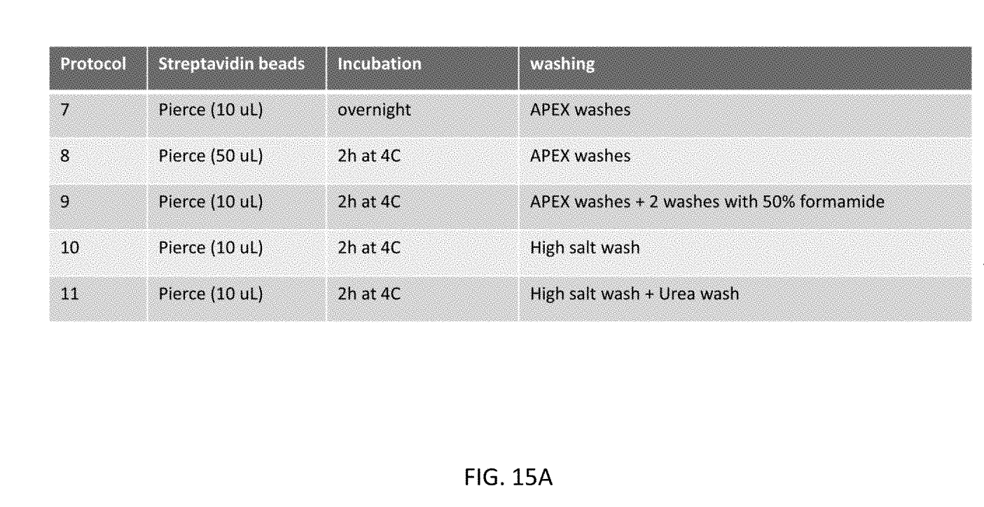

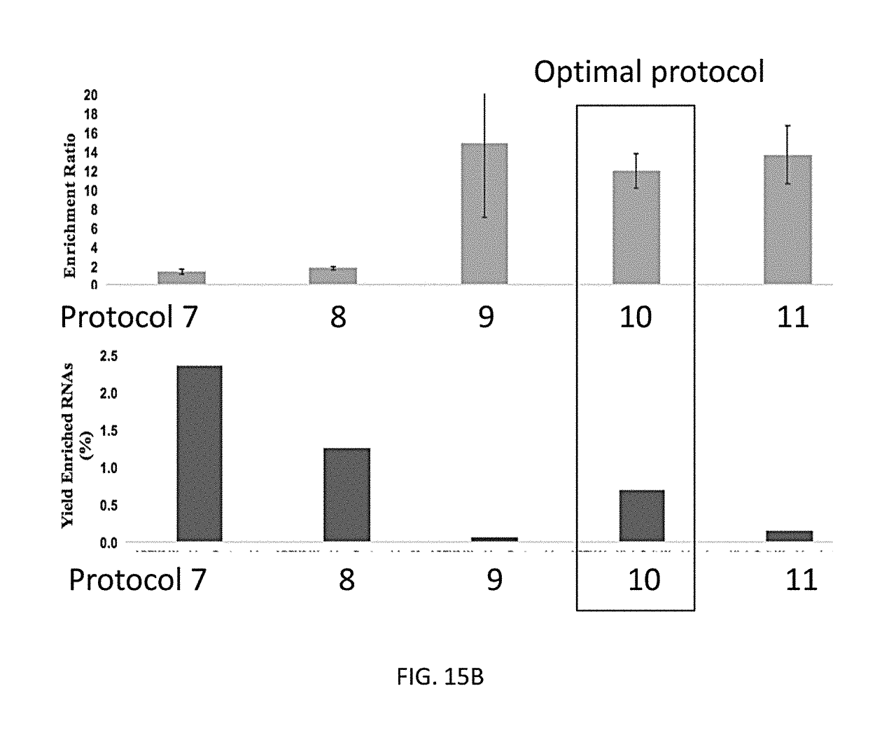

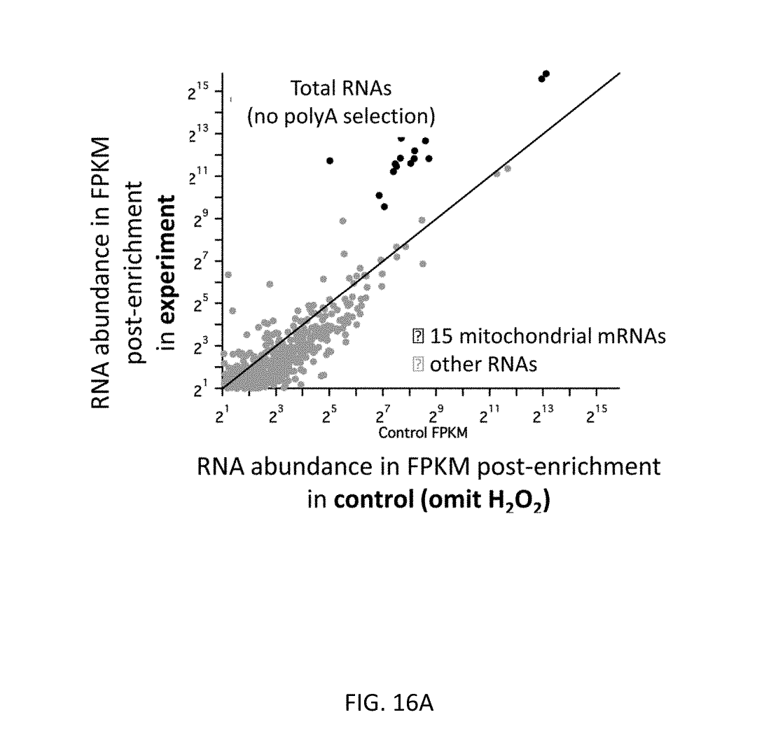

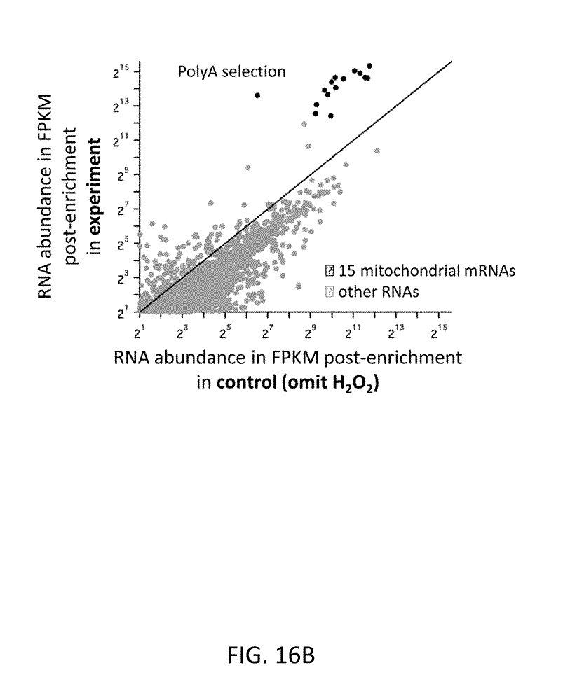

Because of the high reactivity of the phenoxyl radical, it labels nucleic acids locally in proximity to it. This enables its use to study RNA or DNA in proximity to a protein of interest (FIG. 1). In other words, we can generate a protein-DNA or protein-RNA map using APEX. This map provides useful information regarding RNA-protein and DNA-protein interactions in a small area within a cell. For example, if APEX is targeted to a polarized region of the cytoplasm, localization of subcellular mRNAs can be interrogated, whereas if APEX is fused to a nuclear localized protein, long noncoding RNA partners of proteins of interest can be revealed.

### Example 3

### Plasmids and Cloning

APEX-fusion constructs were generated using standard restriction enzyme-based, Gibson assembly, or standard QuikChange methods. All the lentiviral constructs were cloned into plx304 vector. The non-lentiviral constructs are cloned into pCDNA3 plasmid. See Table 1.

TABLE 1
Genetic constructs used in this study

| Name | Features | Promoter/Vector | Details |
|---|---|---|---|
| mito-V5-APEX2 | mito-BamHI-V5- | CMV/pLX304 | Mito is a 24-amino acid |
| | APEX2-NheI | | mitochondrial targeting |
| | | | sequence (MTS) derived |
| | | | from COX4. |
| | | | V5: GKPIPNPLLGLDST |
| | | | (SEQ ID NO: 1) |
| V5-APEX2-NLS | NotI-V5-APEX2- | CMV | NLS: DPKKKRKV |
| | EcoRI-3xNLS-NheI | | (SEQ ID NO: 2) |
| FLAG-APEX2-NES | BstBI-FLAG- | CMV/pLX304 | NES: LQLPPLERLTLD |
| | APEX2-NES-XhoI | | (SEQ ID NO: 3) |
| ERM-APEX2-V5 | BstBI-ERM- | CMV/pLX304 | ERM is ER membrane |
| | APEX2-V5-NheI | | targeting sequence |
| | | | derived from N-terminal |
| | | | 27 amino acids of |
| | | | rabbit P450 |
| | | | C1: MDPVVVLGLCLSCLLLLSL |
| | | | WKQSYGGG |
| | | | (SEQ ID NO: 4) |
| OMM-APEX2 | FLAG-APEX2-MAVS | CMV/pLX304 | MAVS is C terminal 31 |
| | | | residues of MAVS: |
| | | | RPSPGALWLQVAVTGVLVVTLLV |
| | | | VLYRRRLH |
| | | | (SEQ ID NO: 5) |

### Example 4

### Mammalian Cell Culture

HEK-293T from ATCC (passages <25) were cultured in a 1:1 DMEM:MEM mixture (Cellgro) supplemented with 10% FBS, 50 units/mL penicillin, and 50 μg/mL streptomycin at 37° C. under 5% CO₂. Mycoplasma testing was not performed before experiments. For fluorescence microscopy imaging experiments, cells were grown on 7×7-mm glass coverslips in 48-well plates. To improve the adherence of HEK-293T cells, we pretreated glass slides with 50 μg/mL fibronectin (Millipore) for 20 minutes at 37° C. before cell plating and washed three times with Dulbecco's phosphate-buffered saline (DPBS), pH 7.4.

### Example 5

### Preparation of Cells Stably Expressing APEX-Fusion Constructs

Human embryonic kidney (HEK) 293T cells were cultured in Minimum Essential Medium (MEM) supplemented with 10% fetal bovine serum, penicillin, and streptomycin at 37° C. under 5% CO₂. To prepare lentivirus, cells were plated on a T25 plate. Each plate of cells was transfected with 2.5 μg of APEX2 fusion plasmid, 0.25 μg VSVG, and 2.25 μg dR8.91 using 10 μl Lipofectamine 2000 (Invitrogen) in MEM (without serum or antibiotics) at ˜70% confluence. VSVG and dR8.91 are lentiviral packaging plasmids (Pagliarini et al., 2008). The cells were transfected for 3 hours. Then, the media was replaced with 2 ml fresh growth media. After 48 hours, the supernatant was collected and filtered through a 0.45 μm syringe filter. The filtered supernatant was used to infect cells immediately. HEK 293T cells were infected at ˜50% confluency, followed by selection with 8 μg/mL blasticidin in growth medium for 7 days before further analysis.

### Example 6

### Biotin Phenol Labeling In-Vitro

The RNAs were incubated with 2.25 μM HRP, 1 mM H₂O₂, and 500 μM BP. After a period of time as indicated, the reaction was quenched with 10 mM sodium azide, 10 mM ascorbate, 5 mM Trolox. The reactions were cleaned up using Zymo RNA clean and concentrator. For peptide or RNA digestion, the purified RNAs were further incubated with 2 mg/mL proteinase K in PBS or 1 mg/mL RNase A in H₂O for 30 minutes at room temperature. The digested reactions were cleaned up using Zymo RNA clean and concentrator.

### Example 7

### Dot Blot Experiments

The labeled RNAs were spotted on nitrocellulose paper. After the spots were dry, the RNAs were crosslinked to the blot by exposing to 254 nM UV for 3 minutes. Then the blot was incubated briefly with PBS-T (0.1% Tween 20 in PBS) before incubated with 1:3000 streptavidin-HRP (Pierce) in PBS-T for 5 minutes at room temperature. The blot was washed three times with 5 min PBS-T. The blot was developed with Clarity reagents from Bio-Rad.

### Example 8

### Southern Blot Experiment

The labeled DNAs were loaded into 10% Novex TBE urea gel. For genomic DNAs, the DNAs were digested with EcoRI and HindIII overnight before loading. The gel was run at constant 200 volts. The gels were soaked in 1:10000 ethidium bromide (10 mg/mL) in TBE buffer (1 M Tris, 1 M boric acid, 0.02 M EDTA pH 8.3) for 15 minutes. The ethidium bromide fluorescence was imaged by fluorescence gel imager. Then the DNAs in the gel were transferred to positively charge nylon blot in 0.5×TBE buffer at constant 25 volts, maximum 1 ampere for 1 hour. The gel was imaged again and the blot was dried at 60 C for 2 hours. The blot was incubated in blocking buffer (0.05% polyvinylpyrrolidone and 0.05% BSA in H₂O). After 1 hour, the blot was incubated with 1:3000 streptavidin-HRP in blocking buffer for 15 minutes at room temperature. Then the blot was washed with washing buffer (10 mM Tris pH 8.0 and 1 mM EDTA) four times. The blot was developed for chemoluminescence using Bio-Rad Clarity reagents.

### Example 9

### RT-Stop Experiment

³²P-end-labeled DNA primers were annealed to 3 μg of 5s RNA by incubating at 95° C. for 2 minutes followed by a step-down cooling (2° C./seconds) to 4° C. To the reaction first-strand buffer, DTT and dNTPs were added according to manufacturer's protocol for superscript III (ThermoFisher Scientific). The reaction was preincubated at 52° C. for 1 minute, then superscript III (2 units/μL final concentration) was added. Extensions were performed at 50° C. for 10 minutes. To the reaction, one microliter of 4 M sodium hydroxide was added and allowed to react for 5 minutes. Ten microliters of Gel Loading Buffer II (Ambion, Inc.) was then added, and complementary DNA (cDNA) extensions were resolved on 8% denaturing (7 M urea) polyacrylamide gels (29:1 acrylamide and bisacrylamide and 1×TBE). cDNA extensions were visualized by phosphorimaging (STORM, Molecular Dynamics).

Reverse Transcription Primer Used for Human 5S rRNA:

(SEQ ID NO: 6)
5′-AAAGCCTACAGCACCCGGTAT-3′

### Example 10

### Biotin Phenol Labeling and Extraction of Labeled DNA and RNA from a Cell Culture

Stable cell line expressing APEX constructs were plated on a 10 cm disk. At more than 90% confluency, the media was replaced with 500 μM BP in cell culture media. The cells were incubated for 30 minutes at 37° C. Then H₂O₂ was spiked in (final concentration 1 mM). After 1 minute, the media was replaced with PBS+quenchers (10 mM ascorbate, 5 mM Trolox, and 10 mM sodium azide) twice. Then the labeled cells were scrapped and pelleted for further analysis.

Whole cell RNAs were extracted from the labeled cell pellet using Qiagen RNEasy mini plus kit. Genomic DNAs were extracted from Qiagen DNeasy kit using the protocol for cell culture.

### Example 11

### Streptavidin Enrichment

Pre-optimized enrichment protocol: 150 μL Dyna MyOne T1 streptavidin magnetic beads per 25 μg RNAs was prewashed three times with binding and washing buffer (5 mM Tris-HCl pH 7.5, 0.5 mM EDTA, 1 M NaCl), then twice in Solution A (0.1 M NaOH and 0.05 M NaCl), and once in Solution B (0.1 M NaCl). The beads were resuspended in Solution B and 20 μg RNAs were added to the suspension. After incubation for 15 minutes at room temperature. The beads were washed four times with binding and washing buffer using magnetic rack.

Post-optimized enrichment protocol: 10 μL of Pierce streptavidin magnetic beads were prewashed twice with standard RIPA lysis buffer and resuspended in 1 mL RIPA buffer. 25 μg of RNAs were added and incubated at 4° C. After 2 hours, the beads were washed four times with binding and washing buffer using magnetic rack.

The enriched RNAs were released from the beads by incubating with 2 mg/mL proteinase K (Ambion), 2% lauryl sarcoside, 10 mM EDTA, 1% Ribolock RNase inhibitor, 5 mM DTT in 100 μL PBS at 42° C. for an hour and 55 OC for an hour. The released RNAs were cleaned up using Zymo RNA clean and concentrator according to manufacture protocol.

### Example 12

### Real Time-PCR Assay

Whole cell RNAs (no enrichment) and enriched RNAs were reverse transcribed using SuperScript III Reverse Transcriptase kit (ThermoFisher Scientific) with random hexamers (ThermoFisher Scientific). The relative quantity of cDNA was measured using SYBR Green PCR master mix (Applied Biosystems) according to manufacturer's protocol. qRT-PCR primer sequences are listed in Table 2. All data were acquired by Roche LightCycler 480 real time PCR instrument and the data was analyzed by Real time PCR Miner website.

TABLE 2
qRT-PCR primers used in this study

| Primer/probe name | Sequence (5′-3′) |
|---|---|
| MT-ND1 forward | CACCTCTAGCCTAGCCGTTT |
| | (SEQ ID NO: 7) |
| MT-ND1 reverse | CCGATCAGGGCGTAGTTTGA |
| | (SEQ ID NO: 8) |
| MT-COX2 forward | AACCAAACCACTTTCACCGC |
| | (SEQ ID NO: 9) |
| MT-COX2 reverse | CGATGGGCATGAAACTGTGG |
| | (SEQ ID NO: 10) |
| GAPDH forward | TTCGACAGTCAGCCGCATCTTCTT |
| | (SEQ ID NO: 11) |
| GAPDH reverse | GCCCAATACGACCAAATCCGTTGA |
| | (SEQ ID NO: 12) |
| XIST forward | CCCTACTAGCTCCTCGGACA |
| | (SEQ ID NO: 13) |
| XIST reverse | ACACATGCAGCGTGGTATCT |
| | (SEQ ID NO: 14) |
| SSR2 forward | GTTTGGGATGCCAACGATGAG |
| | (SEQ ID NO: 15) |
| SSR2 reverse | CTCCACGGCGTATCTGTTCA |
| | (SEQ ID NO: 16) |
| TMX1 forward | ACGGACGAGAACTGGAGAGA |
| | (SEQ ID NO: 17) |
| TMX1 reverse | ATTTTGACAAGCAGGGCACC |
| | (SEQ ID NO: 18) |
| SFT2D2 forward | CCATCTTCCTCATGGGACCAG |
| | (SEQ ID NO: 19) |
| SFT2D2 reverse | GCAGAACACAGGGTAAGTGC |
| | (SEQ ID NO: 20) |
| FAU forward | TCCTAAGGTGGCCAAACAGG |
| | (SEQ ID NO: 21) |
| FAU reverse | GTGGGCACAACGTTGACAAA |
| | (SEQ ID NO: 22) |
| SUB1 forward | CGTCACTTCCGGTTCTCTGT |
| | (SEQ ID NO: 23) |
| SUB1 reverse | TGATTTAGGCATCGCTTCGC |
| | (SEQ ID NO: 24) |
| IARS2 forward | ACTAGTGGAACAACACGGCA |
| | (SEQ ID NO: 25) |
| IARS2 reverse | GGCCACCAACCTCAGATAAGA |
| | (SEQ ID NO: 26) |
| POLG forward | GAGCAGCAGACCGGGAA |
| | (SEQ ID NO: 27) |
| POLG reverse | CTCTCCTGTCAGTGAAATGGGT |
| | (SEQ ID NO: 28) |
| LARS forward | CCAGGGTCATTGTCGTGGAT |
| | (SEQ ID NO: 29) |
| LARS reverse | AGTCCACTTTGGCTGTTCCT |
| | (SEQ ID NO: 30) |
| LARS2 forward | TCGCCTAAAGGTGGAGAACG |
| | (SEQ ID NO: 31) |
| LARS2 reverse | AGAAGGCCCTGCTCACAG |
| | (SEQ ID NO: 32) |
| MRPL27 forward | ACAGCCGTTACATCCTTGCT |
| | (SEQ ID NO: 33) |
| MRPl27 reverse | CCCGACTTCTTGGATGCGTA |
| | (SEQ ID NO: 34) |
| TIMM44 forward | CGAGGCCAGAAGGCTAGAAG |
| | (SEQ ID NO: 35) |
| TIMM44 reverse | GCACGGTTTCTGACTCGATG |
| | (SEQ ID NO: 36) |

### Example 13

### Library Preparation and Sequencing Analysis

The library was prepared using the TruSeq RNA sample preparation kit, v2 (Illumina) as described in manufacture protocol. The rRNAs were not depleted unless indicated. The indexed libraries were pooled together and sequenced by Illumina HiSeq 2000. For characterization of gene expression, sequencing reads were mapped to a custom gene set comprising UCSC known human genes (hg19) using TopHat2 with default options. Differential analysis of gene expression was assessed using Cuffdiff2 with default options.

### Example 14

### Transfection and Immunofluorescence Staining

To transfect the plasmids, cells plated on 7×7-mm glass coverslips in 48-well plates were transfected at ˜50-60% confluency with 150 ng of the corresponding plasmids and 1 μL of Lipofectamine 2000 for 3 hours. 24 hours after transfection, cell were fixed with 4% paraformaldehyde in PBS at room temperature for 10 minutes. Cells were then washed with PBS three times and permeabilized with cold methanol at −20° C. for 5 minutes. Cells were washed again three times with PBS. Cells were then incubated with primary antibodies in 1% BSA in PBS for 1 hour at room temperature. After washing three times with PBS, cells were incubated with secondary antibodies in 1% BSA in PBS for 30 minutes. Cells were then washed three times with PBS and imaged by confocal microscope.

### Example 15

### Gels and Western Blots

HEK 293T cells stably expressing the indicated constructs were plated in 6-well plates. After labeling, the cells were scraped and pelleted by centrifugation at 3,000 g for 10 minutes. The pellet was stored at −80° C. and then lysed with RIPA lysis buffer (50 mM Tris, 150 mM NaCl, 0.1% SDS, 0.5% sodium deoxycholate, 1% Triton X-100, lx protease cocktail (Sigma Aldrich), 1 mM PMSF (phenylmethylsulfonyl fluoride), for 5 min at 4° C. The cell pellet was resuspended by gentle pipetting. Lysates were clarified by centrifugation at 15,000 g for 10 minutes at 4° C. before separation on a SDS-PAGE gel. Gels were transferred to nitrocellulose membrane, stained by Ponceau S (10 minutes in 0.1% (w/v) Ponceau S in 5% acetic acid/water). The blots were then blocked and stained with primary and secondary antibodies.

While the preferred embodiments of the invention have been illustrated and described, it will be appreciated that various changes can be made therein without departing from the spirit and scope of the invention.

What is claimed is:

1. A method of mapping subcellular localization of nucleic acids in a cell, the method comprising:
a) introducing a tagging enzyme into the cell, wherein the tagging enzyme is targeted to a subcellular region of interest;
b) contacting the cell with a tagging substrate for the tagging enzyme, wherein the tagging enzyme catalyzes a reaction with the tagging substrate resulting in covalent attachment of a tag to nucleic acids within an intracellular spatial location around the tagging enzyme; and
c) isolating the tagged nucleic acids using an agent that selectively binds to the tag; and
d) analyzing the tagged nucleic acids to produce a map of the subcellular localization of the nucleic acids.

2. The method of claim 1, wherein the nucleic acids are RNA or DNA.

3. The method of claim 1, wherein the tagging enzyme is a peroxidase.

4. The method of claim 3, wherein the peroxidase is a horseradish peroxidase or an ascorbate peroxidase.

5. The method of claim 4, wherein the ascorbate peroxidase is APEX or APEX2.

6. The method of claim 3, further comprising contacting the cell with hydrogen peroxide.

7. The method of claim 4, wherein the tagging substrate is biotin-phenol or a derivative thereof.

8. The method of claim 7, wherein the tagging substrate is O-acetylated biotin-phenol.

9. The method of claim 7, wherein said tagging of the nucleic acids comprises reaction of the biotin-phenol or derivative thereof with the hydrogen peroxide to produce a biotin-phenoxyl radical that reacts with nearby nucleic acids resulting in biotinylation of said nucleic acids.

10. The method of claim 1, wherein the tag is biotin and biotinylated nucleic acids are isolated by binding to a biotin-binding protein.

11. The method of claim 10, wherein the biotin-binding protein is streptavidin or avidin.

12. The method of claim 1, further comprising treating the cell with a radical quencher after said tagging of the nucleic acids.

13. The method of claim 12, wherein the radical quencher is ascorbate, 6-hydroxy-2,5,7,8-tetramethylchroman-2-carboxylic acid (TROLOX), or sodium azide.

14. The method of claim 1, wherein the tagging enzyme comprises a targeting sequence that directs the tagging enzyme to the subcellular region of interest.

15. The method of claim 14, wherein the targeting sequence is selected from the group consisting of a secretory protein signal sequence, a membrane protein signal sequence, a nuclear localization sequence, a mitochondrial localization sequence, an outer mitochondrial membrane sequence, an endoplasmic reticulum localization sequence, an endoplasmic reticulum membrane targeting sequence, a nucleolar localization signal sequence, a nuclear export signal sequence, a peroxisome localization sequence, and a protein binding motif sequence.

16. The method of claim 15, wherein the targeting sequence comprises a sequence selected from the group consisting of SEQ ID NOS:1-5.

17. The method of claim 1, wherein the tagging enzyme is covalently linked to a peptide or protein that directs the tagging enzyme to the subcellular region of interest.

18. The method of claim 17, wherein the protein is a cytosolic protein, a nuclear protein, a membrane protein, a mitochondrial protein, a P-body protein, or a secretory pathway protein.

19. The method of claim 1, wherein said introducing the tagging enzyme into the cell comprises transfecting the cell with a recombinant polynucleotide comprising a promoter operably linked to a polynucleotide encoding the tagging enzyme.

20. The method of claim 19, wherein the recombinant polynucleotide comprises a plasmid or viral vector.

21. The method of claim 20, wherein the viral vector is a lentivirus vector.

22. The method of claim 1, further comprising identifying at least one ribonucleoprotein (RNP) interaction.

23. The method of claim 1, further comprising sequencing at least one RNA or DNA molecule in the tagged nucleic acids.

24. The method of claim 1, further comprising multiplex sequencing of the tagged nucleic acids.

25. The method of claim 24, wherein said sequencing comprises performing deep sequencing or next-generation sequencing.

26. The method of claim 1, further comprising calculating the frequencies of one or more RNA molecules that are present within the intracellular spatial location or quantitating one or more RNA molecules that are present within the intracellular spatial location.

27. The method of claim 1, further comprising identifying at least one RNA or DNA molecule of the tagged nucleic acids.

28. The method of claim 27, wherein said at least one RNA is selected from the group consisting of a messenger RNA, a ribosomal RNA, a transfer RNA, a non-coding RNA, and a regulatory RNA.

29. The method of claim 1, wherein the cell is exposed to a test condition prior to said contacting the cell with the tagging substrate.

30. The method of claim 29, wherein the test condition comprises exposing the cell to a drug, a ligand for a receptor, a hormone, a second messenger, a pathogen, or a genetic modification.

31. The method of claim 30, wherein the genetic modification comprises introduction of a vector, short hairpin RNA (shRNA), small interfering RNA (siRNA), microRNA (miRNA), or CRISPR-associated system into the cell.

32. The method of claim 29, wherein the test condition comprises exposing the cell to a change in temperature, growth media, membrane potential, or osmotic pressure.

33. The method of claim 29, wherein a map of the subcellular localization of the RNA molecules within the intracellular spatial location is compared to a reference map for a cell that is not exposed to the test condition.

34. The method of claim 1, wherein a map of the subcellular localization of the nucleic acid molecules within the intracellular spatial location is compared to a reference map for a cell at a different developmental stage.

35. The method of claim 1, wherein the cell is a eukaryotic cell, a prokaryotic cell, or an archaeon cell.

36. The method of claim 35, wherein the cell is an animal cell, a plant cell, a fungal cell, or a protist cell.

37. The method of claim 1, wherein the nucleic acids are RNA selected from the group consisting of animal RNA, bacterial RNA, fungal RNA, protist RNA, plant RNA, and viral RNA.

38. The method of claim 1, wherein the cell is an artificial cell encapsulating the nucleic acids.

39. The method of claim 38, wherein the artificial cell comprises a nanoparticle, liposome, polymersome, or microcapsule.

40. The method of claim 1, wherein the cell is a human cell.

41. The method of claim 1, further comprising amplifying at least one RNA.

42. The method of claim 41, wherein said amplifying comprises performing reverse transcription polymerase chain reaction (RT-PCR).

43. The method of claim 1, further comprising lysing the cell.

44. The method of claim 1, wherein the agent that selectively binds to the tag is selected from the group consisting of an antibody, a probe, a ligand, or an aptamer.

45. The method of claim 44, wherein the agent is immobilized on a solid support.


SEQUENCE LISTING (36 sequences)

SEQ ID NO: 1 - protein, 14 aa, Artificial Sequence
  mitochondrial targeting sequence
GKPIPNPLLGLDST

SEQ ID NO: 2 - protein, 8 aa, Artificial Sequence
  nuclear localization sequence
DPKKKRKV

SEQ ID NO: 3 - protein, 12 aa, Artificial Sequence
  nuclear export signal sequence
LQLPPLERLTLD

SEQ ID NO: 4 - protein, 27 aa, Artificial Sequence
  endoplasmic reticulum membrane targeting sequence
MDPVVVLGLCLSCLLLLSLWKQSYGGG

SEQ ID NO: 5 - protein, 31 aa, Artificial Sequence
  an outer mitochondrial membrane sequence
RPSPGALWLQVAVTGVLVVTLLVVLYRRRLH

SEQ ID NO: 6 - DNA, 21 bp, Artificial Sequence
  reverse transcription primer used for human 5S rRNA
aaagcctacagcacccggtat

SEQ ID NO: 7 - DNA, 20 bp, Artificial Sequence
  MT-ND1 forward primer
cacctctagcctagccgttt

SEQ ID NO: 8 - DNA, 20 bp, Artificial Sequence
  MT-ND1 reverse primer
ccgatcagggcgtagtttga

SEQ ID NO: 9 - DNA, 20 bp, Artificial Sequence
  MT-COX2 forward primer
aaccaaaccactttcaccgc

SEQ ID NO: 10 - DNA, 20 bp, Artificial Sequence
  MT-COX2 reverse primer
cgatgggcatgaaactgtgg

SEQ ID NO: 11 - DNA, 24 bp, Artificial Sequence
  GAPDH forward primer
ttcgacagtcagccgcatcttctt

SEQ ID NO: 12 - DNA, 24 bp, Artificial Sequence
  GAPDH reverse primer
gcccaatacgaccaaatccgttga

SEQ ID NO: 13 - DNA, 20 bp, Artificial Sequence
  XIST forward primer
ccctactagctcctcggaca

SEQ ID NO: 14 - DNA, 20 bp, Artificial Sequence
  XIST reverse primer
acacatgcagcgtggtatct

SEQ ID NO: 15 - DNA, 21 bp, Artificial Sequence
  SSR2 forward primer
gtttgggatgccaacgatgag

SEQ ID NO: 16 - DNA, 20 bp, Artificial Sequence
  SSR2 reverse primer
ctccacggcgtatctgttca

SEQ ID NO: 17 - DNA, 20 bp, Artificial Sequence
  TMX1 forward primer
acggacgagaactggagaga

SEQ ID NO: 18 - DNA, 20 bp, Artificial Sequence
  TMX1 reverse primer
attttgacaagcagggcacc

SEQ ID NO: 19 - DNA, 21 bp, Artificial Sequence
  SFT2D2 forward primer
ccatcttcctcatgggaccag

SEQ ID NO: 20 - DNA, 20 bp, Artificial Sequence
  SFT2D2 reverse primer
gcagaacacagggtaagtgc

SEQ ID NO: 21 - DNA, 20 bp, Artificial Sequence
  FAU forward primer
tcctaaggtggccaaacagg

SEQ ID NO: 22 - DNA, 20 bp, Artificial Sequence
  FAU reverse primer
gtgggcacaacgttgacaaa

SEQ ID NO: 23 - DNA, 20 bp, Artificial Sequence
  SUB1 forward primer
cgtcacttccggttctctgt

SEQ ID NO: 24 - DNA, 20 bp, Artificial Sequence
  SUB1 reverse primer
tgatttaggcatcgcttcgc

SEQ ID NO: 25 - DNA, 20 bp, Artificial Sequence
  IARS2 forward primer
actagtggaacaacacggca

SEQ ID NO: 26 - DNA, 21 bp, Artificial Sequence
  IARS2 reverse primer
ggccaccaacctcagataaga

SEQ ID NO: 27 - DNA, 17 bp, Artificial Sequence
  POLG forward primer
gagcagcagaccgggaa

SEQ ID NO: 28 - DNA, 22 bp, Artificial Sequence
  POLG reverse primer
ctctcctgtcagtgaaatgggt

SEQ ID NO: 29 - DNA, 20 bp, Artificial Sequence
  LARS forward primer
ccagggtcattgtcgtggat

SEQ ID NO: 30 - DNA, 20 bp, Artificial Sequence
  LARS reverse primer
agtccactttggctgttcct

SEQ ID NO: 31 - DNA, 20 bp, Artificial Sequence
  LARS2 forward primer
tcgcctaaaggtggagaacg

SEQ ID NO: 32 - DNA, 18 bp, Artificial Sequence
  LARS2 reverse primer
agaaggccctgctcacag

SEQ ID NO: 33 - DNA, 20 bp, Artificial Sequence
  MRPL27 forward primer
acagccgttacatccttgct

SEQ ID NO: 34 - DNA, 20 bp, Artificial Sequence
  MRPl27 reverse primer
cccgacttcttggatgcgta

SEQ ID NO: 35 - DNA, 20 bp, Artificial Sequence
  TIMM44 forward primer
cgaggccagaaggctagaag

SEQ ID NO: 36 - DNA, 20 bp, Artificial Sequence
  TIMM44 reverse primer
gcacggtttctgactcgatg